(19) United States
(12) Patent Application Publication
(10) Pub. No.: US 2022/0008401 A1
(43) Pub. Date: Jan. 13, 2022

(54) COMPOSITIONS AND METHODS FOR TREATING PULMONARY DISEASE WITH MATRIX METALLOPROTEINASE INHIBITORS

(71) Applicant: DARSHA LLC, New York, NY (US)

(72) Inventor: Jeanine D'Armiento, New York, NY (US)

(21) Appl. No.: 17/423,545

(22) PCT Filed: Jan. 29, 2020
(86) PCT No.: PCT/US2020/015668

§ 371 Date: Jul. 16, 2021

Related U.S. Application Data
(60) Provisional application No. 62/798,069, filed on Jan. 29, 2019.

(51) Int. Cl. A61K 31/4406; A61K 9/00; A61P 11/00; A61P 29/00

(52) U.S. Cl. CPC A61K 31/4406; A61P 29/00; A61P 11/00; A61K 9/0078

(57) ABSTRACT

Disclosed in certain embodiments is a method of treating a pulmonary disease comprising administering a therapeutically effective amount of a Matrix Metalloproteinase (MMP) Inhibitor to a patient in need thereof wherein the pulmonary disease is selected from the group consisting of Acute Respiratory Distress Syndrome, Acute Lung Injury and Acute Inflammatory Injury, and compositions thereof.

The present invention relates to the field of pharmaceuticals for treating pulmonary disease. Specifically, the present invention relates to the use of matrix metalloproteinase inhibitors for the treatment of, e.g., acute respiratory distress syndrome, acute lung injury and acute inflammatory injury.

### BACKGROUND OF THE INVENTION

Acute respiratory distress syndrome (ARDS) is a life-threatening condition typically experienced by a mechanically ventilated patient in an intensive care unit. Typically, pulmonary edema and poor oxygenation result from massive inflammatory damage to the lungs. ARDS arises from a diverse set of pathologies ranging from viral or bacterial insults, inhaled irritants, autoimmune conditions, and trauma. When examined together, despite seemingly discordant etiologies, lung injury ultimately results from the activation of similar signaling pathways in each condition. The early stages of ARDS are characterized by the infiltration of neutrophils (PMN), which release of a myriad of pro-inflammatory cytokines, proteases and free radicals exacerbating lung permeability, leukocyte chemotaxis, and pulmonary injury. As such, trans-pulmonary migration of PMNs has become a marker of disease activity and correlates with the extent of lung injury.

Typical treatment of ARDS includes improving oxygen levels in the blood by supplemental oxygen and managing the amount of fluids (too much fluid can increase fluid buildup in the lungs and too little fluid can put a strain on the heart and other organs and lead to shock).

There is a continued need in the art to develop a pharmaceutical treatment for pulmonary disease such as acute respiratory distress syndrome, acute lung injury and acute inflammatory injury.

### OBJECTS AND SUMMARY OF THE INVENTION

It is an object of certain embodiments of the present invention to provide a pharmaceutical composition comprising an active agent comprising a matrix metalloproteinase inhibitor for the treatment of pulmonary disease.

It is an object of certain embodiments of the present invention to provide a pharmaceutical composition comprising a matrix metalloproteinase inhibitor for the treatment of acute respiratory distress syndrome.

It is an object of certain embodiments of the present invention to provide a pharmaceutical composition comprising a matrix metalloproteinase inhibitor for the treatment of acute lung injury.

It is an object of certain embodiments of the present invention to provide a pharmaceutical composition comprising a matrix metalloproteinase inhibitor for the treatment of acute inflammatory injury. It is an object of certain embodiments of the present invention to provide a method for treating a pulmonary disease with an active agent comprising a matrix metalloproteinase inhibitor.

It is an object of certain embodiments of the present invention to provide a method for treating acute respiratory distress syndrome with a matrix metalloproteinase inhibitor.

It is an object of certain embodiments of the present invention to provide a method for treating acute lung injury with a matrix metalloproteinase inhibitor.

It is an object of certain embodiments of the present invention to provide a method for treating acute inflammatory injury with a matrix metalloproteinase inhibitor. It is an object of certain embodiments of the present invention to provide a matrix metalloproteinase inhibitor compound useful for the treatment of pulmonary disease. The above objects of the present invention and others may be achieved by the present invention which in certain embodiments is directed to a method of treating a pulmonary disease or condition comprising administering a therapeutically effective amount of a matrix metalloproteinase inhibitor to a patient in need thereof wherein the pulmonary disease is selected from the group consisting of acute respiratory distress syndrome, acute lung injury and acute inflammatory injury.

In other embodiments, the present invention is directed to a pharmaceutical composition comprising an effective amount of a matrix metalloproteinase inhibitor to treat acute respiratory distress syndrome, acute lung injury or acute inflammatory injury and a pharmaceutically acceptable excipient suitable for pulmonary administration.

In certain embodiments, the matrix metalloproteinase inhibitor is a compound of Formula I:

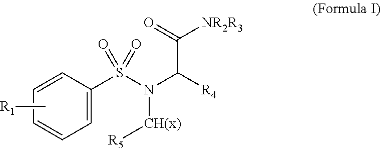

wherein X is an integer from 0-3;

R₁ is 0-3 substitutions independently selected from halogen, hydroxyl, or C₁₋₃ alkyl;

R₂ and R₃ are independently H, hydroxyl or straight or branched C₁₋₃ alkyl;

R₄ is straight o branched C₁₋₅ alkyl; and

R₅ is a mono or bicyclic aromatic or heteroaromatic;

or pharmaceutically acceptable salt or solvate thereof.

The present invention is also directed to all variations, enantiomers and stereoisomers of the compounds disclosed herein.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 (A) depicts the nebulizer used in the present examples and (B) demonstrates that inhaled Compound 1 is successfully absorbed by the mouse lung. In this test, mice received inhaled Compound 1 (10 mg in 4 mL sterile PBS) and were sacrificed at 2, 4 and 6 hours following treatment. Lung tissue was then assayed for Compound 1 content by mass spectroscopy.

FIG. 2 demonstrates that inhaled Compound 1 inhibits MMP 9 activity in LPS treated mice. (A) shows that MMP 9 activity in BALF is decreased in Compound 1 treated mice. (B) shows that MMP 9 activity is decreased in lung tissue in Compound 1 treated animals.

FIG. 3 depicts decreased inflammatory scores of lung slices from LPS injured animals treated with inhaled Compound 1.

FIG. 4 demonstrates significantly reduced edema in LPS injured animals treated with inhaled Compound 1 as assessed by; (A) FITC-albumin BALF:serun ratios, (B) wet:dry ratios.

FIG. 5 demonstrates that the inhalation of Compound 1 compound attenuates apoptosis in the LPS-induced lung injury model as assessed by western blot (A and B), caspase 3/7 activity assay (C), and the fluorescently stained lung tissue sections depicting cleaved caspase-3 and cell nuclei of a PBS-PBS treated mouse, a LPS-PBS treated mouse, and a LPS-CGS treated mouse (D).

FIG. 6 demonstrates that Compound 1 compound reduces PMN counts in murine BALF in the LPS-induced lung injury model. (A) total and PMN cell counts from BALF. (B) Representative images of stained BALF.

FIG. 7 demonstrates decreased PMN present in the lung parenchyma of LPS injured mice treated with inhaled Compound 1 as assessed by myeloperoxidase staining.

FIG. 8, (A) demonstrates that inhaled Compound 1 is absorbed by the lungs at a concentration independent manner and at a time dependent manner. (B) demonstrates an alternative treatment paradigm for mice receiving inhaled Compound 1.

FIG. 9, (A) representative H&E stained paraffin embedded sections qualitatively revealing reduced inflammation in compound 1 treated animals as compared to mice receiving only LPS. (B) Lung injury scoring system from Matute-Bell et al (2008). (C) Lung Injury Score (LIS) from experimental mice.

FIG. 10, (A) demonstrates that inhaled Compound 1 treatment attenuates LPS-induced edema as demonstrated by the reduced wet:dry weight ratio in the LPS-CGS group as compared to the LPS-PBS group. (B) demonstrates that inhaled Compound 1 treatment attenuates LPS-induced vascular permeability as demonstrated by the reduced BAL:serum FTIC Albumin ratio in the LPS-CGS group as compared to the LPS-PBS group.

FIG. 11, (A) representative myeloperoxidase stained paraffin embedded sections qualitatively revealing reduced neutrophils in Compound 1 treated animals (post LPS injury) as compared to mice receiving only LPS. (B) Representative DifQuick stains of cytospins from BALF showing a decrease in neutrophils following treatment of LPS-injured mice with inhaled Compound 1. (C) Differential cell counts taken from each animal's cytospin with at least three separate images counted. (D) A Quantikine Assay quantifying the inhibition of MMP-9 by inhaled Compound 1 in LPS-injured mice.

FIG. 12, (A) A quantification of BrdU(+) neutrophils and BrdU(−) neutrophils demonstrating that Compound 1 treatment inhibits LPS-induced influx of newly synthesized neutrophils from the vascular pool. (B) Representative immunofluorescent images for stained cytospins showing BrdU stained neutrophils and macrophages.

FIG. 13, (A) Schematic for migration experiments in a modified Boyden chamber. (B) Depicts that FMLP treatment resulted in significant increase in neutrophil migration into the basal chamber as compared to Compound 1 treated neutrophils and vehicle controls. (C) Demonstrates that Compound 1 treatment has no significant effects on neutrophil viability in vitro.

FIG. 14, (A) Representative images demonstrating decreased 01 (green) expression by Compound 1 treated neutrophils. (B) Quantification of fluorescent integrin β1 signal intensity as compared to the number of neutrophils present in each field.

FIG. 15 demonstrates that Compound 1 effects are specific to a model of acute lung inflammation and are not observed in more chronic model of pulmonary injury.

### DEFINITIONS

As used herein, the singular forms “a,” “an,” and “the” include plural references unless the context clearly indicates otherwise. Thus, for example, reference to “an excipient” includes a single excipient as well as a mixture of two or more different excipients, and the like.

As used herein, the term “about” in connection with a measured quantity, refers to the normal variations in that measured quantity, as expected by one of ordinary skill in the art in making the measurement and exercising a level of care commensurate with the objective of measurement and the precision of the measuring equipment. In certain embodiments, the term “about” includes the recited number ±10%, such that “about 10” would include from 9 to 11.

As used herein, the term “active agent” refers to any material that is intended to produce a therapeutic, prophylactic, or other intended effect, whether or not approved by a government agency for that purpose. These terms with respect to specific agents include all pharmaceutically active agents, all pharmaceutically acceptable salts thereof, complexes, stereoisomers, crystalline forms, co-crystals, ether, esters, hydrates, solvates, and mixtures thereof, where the form is pharmaceutically active.

As used herein, the term “variations” refers to a compound's various isomers thereof, various structural modifications thereof, and combinations thereof.

As used herein, the term “stereoisomers” is a general term for all isomers of individual molecules that differ only in the orientation of their atoms in space. It includes enantiomers and isomers of compounds with one or more chiral centers that are not mirror images of one another(diastereomers).

The term “enantiomer” or “enantiomeric” refers to a molecule that is nonsuperimposable on its mirror image and hence optically active wherein the enantiomer rotates the plane of polarized light in one direction by a certain degree, and its mirror image rotates the plane of polarized light by the same degree but in the opposite direction.

The term “chiral center” refers to a carbon atom to which four different groups are attached.

The term “patient” refers to a subject, an animal or a human, who has presented a clinical manifestation of a particular symptom or symptoms suggesting the need for treatment, who is treated preventatively or prophylactically for a condition, or who has been diagnosed with a condition to be treated. The term “subject” is inclusive of the definition of the term “patient” and does not exclude individuals who are otherwise healthy.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

The term “condition” or “conditions” refers to those medical conditions that can be treated or prevented by administration to a subject of an effective amount of the pharmaceutical composition disclosed herein, e.g., acute respiratory distress syndrome, acute lung injury, acute inflammatory injury, and the like.

The terms “treatment of” and “treating” includes the lessening of the severity of or cessation of a condition or lessening the severity of or cessation of symptoms of a condition.

The terms “prevention of” and “preventing” includes the avoidance of the onset of a condition.

“Therapeutically effective amount” is intended to include an amount of an active agent, or an amount of the combination of active agents, to treat or prevent the condition, to treat the symptoms of the condition, in a subject.

The phrase “pharmaceutically acceptable” refers to those compounds, materials, compositions, and/or dosage forms that are, within the scope of sound medical judgment, suitable for use in contact with the tissues of human beings and animals without excessive toxicity, irritation, allergic response, or other problems or complications commensurate with a reasonable benefit/risk ratio.

The term “salt” includes non-toxic pharmaceutically acceptable salts. Examples of pharmaceutically acceptable addition salts include inorganic and organic acid addition salts and basic salts. The pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. Acid addition salts can be formed by mixing a solution of the particular compound with a solution of a pharmaceutically acceptable non-toxic acid such as hydrochloric acid, fumaric acid, maleic acid, succinic acid, acetic acid, citric acid, tartaric acid, carbonic acid, phosphoric acid, oxalic acid, dichloroacetic acid, or the like. Basic salts can be formed by mixing a solution of the compound of the present disclosure with a solution of a pharmaceutically acceptable non-toxic base such as sodium hydroxide, potassium hydroxide, choline hydroxide, sodium carbonate and the like.

In certain embodiments, the present invention encompasses the preparation and use of solvates of compounds used in the invention. Solvates typically do not significantly alter the physiological activity or toxicity of the compounds, and as such may function as pharmacological equivalents. The term “solvate” as used herein is a combination, physical association and/or solvation of a compound of the present invention with a solvent molecule such as, e.g. a disolvate, monosolvate or hemisolvate, where the ratio of solvent molecule to compound of the present disclosure is about 2:1, about 1:1 or about 1:2, respectively. This physical association involves varying degrees of ionic and covalent bonding, including hydrogen bonding. In certain instances, the solvate can be isolated, such as when one or more solvent molecules are incorporated into the crystal lattice of a crystalline solid. Thus, “solvate” encompasses both solution-phase and isolatable solvates. Compounds disclosed herein can be present as solvated forms with a pharmaceutically acceptable solvent, such as water, methanol, ethanol, and the like, and it is intended that the disclosure includes both solvated and unsolvated forms of these compounds. One type of solvate is a hydrate. A “hydrate” relates to a particular subgroup of solvates where the solvent molecule is water. Solvates typically can function as pharmacological equivalents. Preparation of solvates is known in the art. See, for example, M. Caira et al, J. Pharmaceut. Sci., 93(3):601-611 (2004), which describes the preparation of solvates of fluconazole with ethyl acetate and with water. Similar preparation of solvates, hemisolvates, hydrates, and the like are described by E. C. van Tonder et al., AAPS Pharm. Sci. Tech., 5(1): Article 12 (2004), and A. L. Bingham et al., Chem. Commun. 603-604 (2001). A typical, non-limiting, process of preparing a solvate would involve dissolving a compound disclosed herein in a desired solvent (organic, water, or a mixture thereof) at temperatures above 20° C. to about 25° C., then cooling the solution at a rate sufficient to form crystals, and isolating the crystals by known methods, e.g., filtration. Analytical techniques such as infrared spectroscopy can be used to confirm the presence of the solvent in a crystal of the solvate.

For the purpose of the present disclosure, the term “aryl” or “aromatic” as used by itself or as part of another group refers to a monocyclic or bicyclic aromatic ring system having from six to fourteen carbon atoms (i.e., C₆₋₁₄ aryl) and also refers to tricyclic ring systems. Non-limiting exemplary aryl groups include phenyl (abbreviated as “Ph”), naphthyl, phenanthryl, anthracyl, indenyl, azulenyl, biphenyl, biphenylenyl, and fluorenyl groups. In one embodiment, the aryl group is chosen from phenyl or naphthyl.

For the purpose of the present disclosure, the term “heteroaryl” or “heteroaromatic” refers to monocyclic and bicyclic aromatic ring systems having 5 to 14 ring atoms (i.e., C₅₋₁₄ heteroaryl) and 1, 2, 3, or 4 heteroatoms independently chosen from oxygen, nitrogen and sulfur. The term also refers to tricyclic ring systems. In one embodiment, the heteroaryl has three heteroatoms. In another embodiment, the heteroaryl has two heteroatoms. In another embodiment, the heteroaryl has one heteroatom. In one embodiment, the heteroaryl is a C₅ heteroaryl. In another embodiment, the heteroaryl is a C₆ heteroaryl. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, pyrimidinyl, phenanthrolinyl, phenazinyl, thiazolyl, isothiazolyl, phenothiazolyl, isoxazolyl, furazanyl, and phenoxazinyl. In one embodiment, the heteroaryl is chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, pyrimidin-5-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) and isoxazolyl (e.g., isoxazol-3-yl, isoxazol-4-yl, and isoxazol-5-yl). The term “heteroaryl” is also meant to include possible N-oxides. Exemplary N-oxides include pyridyl N-oxide and the like.

### DETAILED DESCRIPTION

Certain embodiments of the invention are directed to a method of treating a pulmonary disease comprising administering a therapeutically effective amount of a Matrix Metalloproteinase (MMP) Inhibitor to a patient in need thereof, wherein the pulmonary disease is selected from the group consisting of Acute Respiratory Distress Syndrome, Acute Lung Injury, Acute Inflammatory Injury, and combinations thereof.

In certain methods of the present invention, the MMP inhibitor is a collagenase (e.g., one or more of MMP-1, MMP-8, MMP-13) inhibitor, gelatinases (e.g., one or more of MMP-2, MMP-9) inhibitor, stromelysins (e.g., one or more of MMP-3, MMP-10, MMP-11) inhibitor, matrilysins (e.g., one or more of MMP-7, MMP-26) inhibitor, membrane-type (e.g., MT) MMPs (e.g., one or more of MMP-14, MMP-15, MMP-16, MMP-17, MMP-24, MMP-25) inhibitor, and other MMPs (e.g., one or more of MMP-12, MMP-19, MMP-20, MMP-21, MMP-23, MMP-27, MMP-28) inhibitor, or a combination thereof.

In certain methods of the present invention, the MMP Inhibitor is a compound of Formula I:

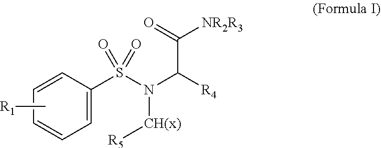

wherein X is an integer from 0-3;

R₁ is 0-3 substitutions independently selected from halogen, hydroxyl or C₁₋₃ alkyl;

R₂ and R₃ are independently H, hydroxyl or straight or branched C₁₋₃ alkyl;

R₄ is straight o branched C₁₋₅ alkyl; and

R₅ is a mono or bicyclic aromatic or heteroaromatic;

or pharmaceutically acceptable salt or solvate thereof.

In certain methods of the present invention, the compound of Formula I is:

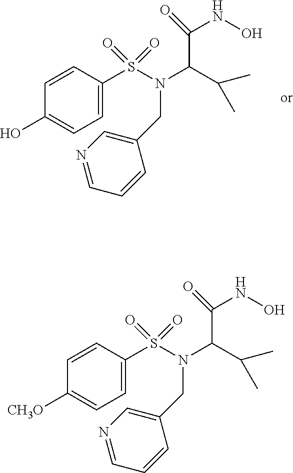

or pharmaceutically acceptable salt thereof, or solvate thereof.

In certain methods of the present invention, the MMP Inhibitor is a compound of Formula I, marimastat (BB-2516), batimastat (BB-94), PD166793, Ro32-3555, WAY170523, UK370106, TIMP1, TIMP2, TIMP3, TIMP4, RS113456, PKF242-484, CP 471,474, AZ11557272, AS112108, AS111793#, MMP408, GM6001 Ilomastat (Galardin®), doxycycline, R-94138, MMPI-I, MMPI-II (MMP2/MMP9 inhibitor II), MMP9 inhibitor I, MMP8 inhibitor I, ONO-4817, COL-3 (matastat), cyclohexylamine salt of (R)-1-(3′-methylbiphenyl-4-sulfonylamino)-methylpropyl phosphonic acid, MMI270, BMS-275291 (rebimastat), BAY 12-9566, SB-3CT, CH1104, or a combination thereof.

In certain methods of the present invention, the administration is pulmonary administration.

In certain methods of the present invention, the pulmonary administration is by oral inhalative administration or intranasal administration.

In certain methods of the present invention, the oral inhalative administration is by intratracheal instillation or intratracheal inhalation with an endotracheal tube.

In certain methods of the present invention, the intratracheal instillation comprises administering a solution or suspension of the MMP inhibitor to the pulmonary system by a syringe.

In certain methods of the present invention, the intratracheal inhalation comprises inhaling an aerosol comprising the MMP inhibitor.

In certain methods of the present invention, the aerosol is delivered by a metered dose inhaler.

In certain methods of the present invention, the intratracheal inhalation comprises inhaling a nebulized solution of the MMP inhibitor.

In certain methods of the present invention, the nebulized solution is delivered by jet nebulizer, ultrasonic nebulizer or vibrating mesh nebulizer.

In certain methods of the present invention, the intratracheal inhalation comprises inhaling a powder comprising the MMP inhibitor.

In certain methods of the present invention, the powder is administered by a dry powder inhaler.

In certain methods of the present invention, the duration of treatment with the pharmaceutical composition is (continuously or intermittently) over a time period, e.g., of up to 30 days, up to 25 days, up to 20 days, up to 15 days, up to 10 days, up to 7 days, up to 6 days, up to 5 days, up to 4 days, up to 3 days, up to 2 days (48 hours), or up to 1 day (24 hours). In certain embodiments, the pharmaceutical composition is administered over a duration that is long enough to effectively treat, minimize, prevent, or inhibit any of the pulmonary disease described herein, yet short enough to minimize side effects that may otherwise be observed with chronic administration of the pharmaceutical composition.

In certain methods of the present invention, the dosing regimen of the pharmaceutical composition is hourly, every two hours, every three hours, every four hours, every 5 hours, four times daily (once every 6 hours), three times daily (once every 8 hours), twice daily (once every 12 hours), once daily, once every 48 hours, once every 72 hours, once every 96 hours, once every 120 hours, once every 144 hours, or once every 168 hours.

In certain embodiments, each administration can be for at least 1 minute, at least 5 minutes, at least 10 minutes, at least 15 minutes, at least 20 minutes, at least 30 minutes, at least 45 minutes, at least 1 hour, at least 2 hours, at least 4 hours, at least 6 hours, at least 8 hours, at least 12 hours, or at least 24 hours as a single treatment or according to the duration of treatment and dosing regimen disclosed herein.

In certain embodiments, a dose of from any of about 0.01 mg/kg, about 0.05 mg/kg, about 0.1 mg/kg, about 0.5 mg/kg, about 1 mg/kg, about 2 mg/kg, about 4 mg/kg, about 6 mg/kg, about 8 mg/kg, about 10 mg/kg, about 15 mg/kg, about 20 mg/kg, about 30 mg/kg, about 40 mg/kg, about 50 mg/kg to any of about 75 mg/kg, about 100 mg/kg, about 150 mg/kg, about 200 mg/kg, about 250 mg/kg, about 300 mg/kg, about 350 mg/kg, about 400 mg/kg, about 450 mg/kg, or about 500 mg/kg, of an MMP inhibitor such as, without limitations, the compound of Formula I (or pharmaceutical acceptable salt thereof or solvate thereof), may be administered to a patient in need thereof, e.g., via pulmonary administration. In other embodiments, the dose is from any of about 0.01 mg/kg, about 0.05 mg/kg, about 0.1 mg/kg, about 0.5 mg/kg, about 1 mg/kg, about 2 mg/kg, about 4 mg/kg, about 6 mg/kg, about 8 mg/kg, about 10 mg/kg to any of about 15 mg/kg, about 20 mg/kg, about 30 mg/kg, about 40 mg/kg, about 50 mg/kg, about 75 mg/kg or about 100 mg/kg. In certain embodiments, the dose administered is high enough to effectively treat, minimize, prevent, or inhibit any of the pulmonary disease described herein, yet low enough to minimize side effects that may otherwise be observed with higher doses

In certain methods of the present invention, administering a pharmaceutical composition as disclosed herein to a patient experiencing a pulmonary disease according to an embodiment may attenuate at least one of neutrophil migration into and out of the lungs, edema, or apoptosis in the lung of that patient.

In certain methods of the present invention, administering a pharmaceutical composition as disclosed herein to a patient experiencing a pulmonary disease according to an embodiment may reduce at least one of inflammation score, edema level, neutrophil count, or caspase activity after administration of the pharmaceutical composition such that the level is substantially similar to that of a healthy subject.

In certain methods of the present invention, administering a pharmaceutical composition as disclosed herein to a patient experiencing a pulmonary disease according to an embodiment increases oxygen level in the blood by up to about 1%, up to about 2%, up to about 3%, up to about 4%, up to about 5%, up to about 6%, up to about 7%, up to about 8%, up to about 9%, up to about 10%, up to about 12%, up to about 14%, up to about 16%, up to about 18%, or up to about 20% compared to baseline. “Baseline” as used herein referring to the oxygen level in the blood of the patient experiencing said pulmonary disease prior to initiation of treatment with the pharmaceutical composition described herein. In other embodiments, the oxygen level in the blood is increased at least about 1%, at least about 2%, at least about 3%, at least about 4%, at least about 5%, at least about 6%, at least about 7%, at least about 8%, at least about 9%, at least about 10%, at least about 12%, at least about 14%, at least about 16%, at least about 18%, or at least about 20% compared to baseline

Certain embodiments of the present invention are directed to a pharmaceutical composition comprising an effective amount of a Matrix Metalloproteinase (MMP) Inhibitor to treating Acute Respiratory Distress Syndrome and a pharmaceutically acceptable excipient suitable for pulmonary administration. In certain embodiments, the pharmaceutical composition is in the form of a solid or a liquid.

In certain embodiments, the pharmaceutical composition is a powder.

In certain embodiments, the pharmaceutical composition is a solution or a suspension of the active agent.

In certain embodiments, the pharmaceutical composition is in a form that is suitable for pulmonary administration. For instance, in certain embodiments the pharmaceutical composition may be in a form of a solid powder, a solution, or a suspension having a particle size ranging from any of about 0.1 μm, about 0.2 μm, about 0.3 μm, about 0.4 μm, about 0.5 μm, about 0.6 μm, about 0.7 μm, about 0.8 μm, about 0.9 μm, or about 1 μm to any of about 2 μm, about 3 μm, about 4 μm, about 5 μm, about 6 μm, about 7 μm, about 8 μm, about 9 μm, about 10 μm, or about 15 μm.

In certain embodiments, the pharmaceutical composition may be administered via pulmonary administration at a flow rate of from any of about 1 lpm, 2 lpm, 4 lpm, 5 lpm, 7 lpm or 10 lpm to any of about 12 lpm, 15 lpm, 17 lpm, 20 lpm or 25 lpm.

In certain embodiments, a single dose of the pharmaceutical composition may have a volume ranging from any of about 0.1 mL, about 0.2 mL, about 0.3 mL, about 0.4 mL, about 0.5 mL, about 0.6 mL, about 0.7 mL, about 0.8 mL, about 0.9 mL, or about 1 mL to any of about 1.5 mL, about 2 mL, about 2.5 mL, about 3.0 mL, about 3.5 mL, about 4.0 mL, about 4.5 mL, about 5.0 mL, about 7.5 mL, about 10 mL, about 15 mL, about 30 mL, about 60 ml, about 90 mL or about 120 mL.

In certain embodiments, the concentration of active agent in the pharmaceutical composition may range from any of about 0.01 mg/mL, about 0.05 mg/mL, about 0.1 mg/mL, about 0.2 mg/mL, about 0.3 mg/mL, about 0.4 mg/mL, about 0.5 mg/mL, about 0.6 mg/mL, about 0.7 mg/mL, about 0.8 mg/mL, about 0.9 mg/mL, about 1.0 mg/mL, about 1.25 mg/mL, about 1.5 mg/mL, about 1.75 mg/mL, about 2.0 mg/mL, about 2.5 mg/mL, or about 3.0 mg/mL to any of about 4 mg/mL, about 4.5 mg/mL, about 5 mg/mL, about 5.5 mg/mL, about 6.0 mg/mL, about 6.5 mg/mL, about 7.0 mg/mL, about 7.5 mg/mL, about 8 mg/mL, about 8.5 mg/mL, about 9.0 mg/mL, about 9.5 mg/mL, about 10.0 mg/mL, about 50 mg/mL or about 100 mg/mL.

In certain embodiments, the MMP inhibitor is a collagenase (e.g., at least one of MMP-1, MMP-8, MMP-13) inhibitor, gelatinases (e.g., at least one of MMP-2, MMP-9) inhibitor, stromelysins (e.g., at least one of MMP-3, MMP-10, MMP-11) inhibitor, matrilysins (e.g., at least one of MMP-7, MMP-26) inhibitor, membrane-type (e.g., MT) MMPs (e.g., at least one of MMP-14, MMP-15, MMP-16, MMP-17, MMP-24, MMP-25) inhibitor, and other MMPs (e.g., at least one of MMP-12, MMP-19, MMP-20, MMP-21, MMP-23, MMP-27, MMP-28) inhibitor, or a combination thereof.

In certain embodiments, the pharmaceutical composition comprises an MMP Inhibitor of Formula I:

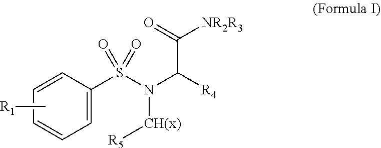

wherein X is an integer from 0-3;

R₁ is 0-3 substitutions independently selected from halogen, hydroxyl or C₁₋₃ alkyl;

R₂ and R₃ are independently H, hydroxyl or straight or branched C₁₋₃ alkyl;

R₄ is straight o branched C₁₋₅ alkyl; and

R₅ is a mono or bicyclic aromatic or heteroaromatic;

or pharmaceutically acceptable salt or solvate thereof.

In certain embodiments, the pharmaceutical composition comprises a compound of Formula I which is:

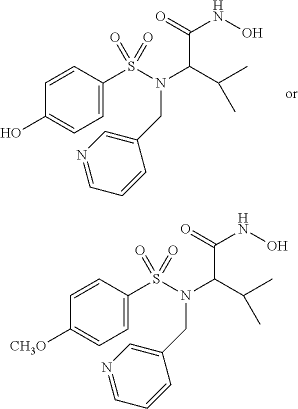

or pharmaceutically acceptable salt thereof or solvate thereof.

In certain embodiments, the pharmaceutical composition comprises an MMP Inhibitor selected from a compound of Formula I, marimastat (BB-2516), batimastat (BB-94), PD166793, Ro32-3555, WAY170523, UK370106, TIMP1, TIMP2, TIMP3, TIMP4, RS113456, PKF242-484, CP 471,474, AZ11557272, AS112108, AS111793#, MMP408, GM6001 Ilomastat (Galardin®), doxycycline, R-94138, MMPI-I, MMPI-II (MMP2/MMP9 inhibitor II), MMP9 inhibitor I, MMP8 inhibitor I, ONO-4817, COL-3 (matastat), cyclohexylamine salt of (R)-1-(3′-methylbiphenyl-4-sulfonylamino)-methylpropyl phosphonic acid, MMI270, BMS-275291 (rebimastat), BAY 12-9566, SB-3CT, CH1104, and a combination thereof.

Certain embodiments of the present invention are directed to a drug delivery system comprising a pharmaceutical composition as disclosed herein contained in a drug delivery device suitable for pulmonary administration.

In certain embodiments, the drug delivery system is suitable for oral inhalative administration or intranasal administration.

In certain embodiments the drug delivery system is suitable for oral inhalative administration selected from intratracheal instillation or intratracheal inhalation.

In certain embodiments the drug delivery device suitable for intratracheal instillation is a syringe and the pharmaceutical composition is in the form of a solution or suspension of the MMP inhibitor.

In certain embodiments the drug delivery device suitable for intratracheal inhalation is a metered dose inhaler suitable to provide an aerosol and the pharmaceutical composition is in the form of a solution or suspension of the MMP inhibitor.

In certain embodiments the drug delivery device suitable for intratracheal inhalation is a nebulizer and the pharmaceutical composition is in the form of a solution or suspension of the MMP inhibitor.

In certain embodiments, the nebulizer is a jet nebulizer, an ultrasonic nebulizer or vibrating mesh nebulizer.

In certain embodiments, the drug delivery device suitable for intratracheal inhalation is a dry powder inhaler and the pharmaceutical composition is in the form of a powder.

In certain embodiments, the present invention is directed to a pharmaceutical composition comprising (i) a compound of Formula I as disclosed above in combination with a pharmaceutically acceptable excipient. The pharmaceutical composition can comprise an MMP inhibitor such as, without limitations, the compound of Formula I (or pharmaceutical acceptable salt thereof or solvate thereof), in an amount (w/w) from about 1% to about 99%, about 10% to about 90%, about 20% to about 80%, about 30% to about 70%, about 40% to about 60% or about 45% to about 55%.

In certain embodiments, the pharmaceutical composition is suitable for oral, sublingual, topical, rectal, pulmonary, intranasal or parenteral administration

In certain embodiments, the pharmaceutical composition is suitable for pulmonary administration wherein the composition is a solution or suspension and is contained in a metered dose inhaler or nebulizer.

In certain embodiments, the pharmaceutical composition is suitable for pulmonary administration wherein the composition is a powder and is contained in a dry powder inhaler.

In certain embodiments, the method comprises administration by a route selected from oral, sublingual, topical, rectal, pulmonary, inhalation, intranasal or parenteral administration.

In an embodiment, the method comprises administration by inhalation or intranasal administration since it could offer several advantages over systemic administration. The advantages include, without limitations, direct delivery to the site of interest (i.e. lungs) and reduced side effects. For inhalation or intranasal administration, the agent can be administered using a nebulizer, inhaler, atomizer, aerosolizer, mister, dry powder inhaler, metered dose inhaler, metered dose sprayer, metered dose mister, metered dose atomizer, or other suitable delivery device.

In some embodiments, the pharmaceutical composition may further comprise a pharmaceutically acceptable excipient. The excipient can be in an amount (w/w) from about 1% to about 99%, about 10% to about 90%, about 20% to about 80%, about 30% to about 70%, about 40% to about 60% or about 45% to about 55%.

The pharmaceutically acceptable excipient may include, without limitations, solvents, suspension mediums, surfactants (e.g., dodecyl b-maltoside), dyes, perfumes, thickening agents, stabilizers, skin penetration enhancers, preservatives, antioxidants, other active agents (e.g., anesthetics or analgesics) and combinations thereof.

The pharmaceutical composition may optionally include one or more preservatives, e.g., methyl hydroxybenzoate, propyl hydroxybenzoate, chlorocresol, benzalkonium chlorides, and the like.

Other suitable excipients may include, for example, starch, glucose, lactose, mannitol, magnesium stearate, talc, cellulose, magnesium carbonate, sodium bicarbonate, citric acid, water, saline solution, aqueous dextrose, glycerol, alcohols (e.g., propylene glycol, phenoxyethanol, methanol, ethanol, isopropyl alcohol, and mixtures thereof) mineral oil, lanolin, gums of vegetable origin, polyalkylene glycols, and the like.

Surfactants useful in the compositions of the present invention include those selected from the group consisting of dodecyl b-maltoside, sarcosinates, dioctyl sodium sulfoscuccinate, pluronic F68, sodium lauryl sulfate, sorbitan monolaurate, lauryldimethylamineoxide, lauric-diethanolamide, PEG-Esters (polyethylene glycol-dilaurate), coconut hydroxyethyl imidazoline, sodium sulfosuccinate ester of lauric MEA, sodium sulfosuccinate ester of ethoxylated lauryl alcohol, lauric-monoethanolamide, bis-(2-hydroxyethyl) cocoamine oxide, polyoxypropylene bases, coconut fatty acid, 2-sulfo-ester, sodium salt, N-coconut oil acyl-N-methyl taurine, sodium salt, lauroyl sarcosine, 30% sodium lauryl sarcosinate, sodium lauroyl sarcosinate, myristoyl sarcosine, oleoyl sarcosine, stearoyl sarcosine, polyoxyethelene 21 stearyl ether (0.1 BHA & 0.005% citric acid as preservatives), lauroamphoglycinate, lauroamphocarboxyglycinate, lauroamphocarboxypropinate, lauroamphocarboxyglycinate-sulfanate, sodium lauryl sulfate (66% lauryl, 27% myristyl, 71% cetyl), polyoxyethylene sorbitan mono-oleate, and mixtures thereof.

### Additional Agents

In certain embodiments, the present invention is directed to pharmaceutical formulations comprising one or more of the MMP inhibitors disclosed herein in combination with an antibiotic. Other embodiments are directed to combination therapy for treating pulmonary diseases or conditions comprising administering one or more of the MMP inhibitors disclosed herein with an antibiotic. The MMP inhibitor can be in the same formulation or a different formulation than the antibiotic. The MMP inhibitor can also be administered by a different route (e.g., oral, nasal, parenteral, inhalation, topical) than the antibiotic. The administration can be before, concurrently or after the administration of the antibiotic.

The term “antibiotic” is used to refer to antibacterial agents that may be derived from bacterial sources. Antibiotic agents may be bactericidal and/or bacteriostatic.

The antibiotic used in combination with the compounds of the present invention may be aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides and tetracycline.

In certain embodiments, the antibiotic may be an aminoglycoside such as Amikacin, Gentamicin, Kanamycin, Neomycin, Netilmicin, Tobramycin or Paromomycin.

In other embodiments, the antibiotic agent may be a carbapenem such as Ertapenem, Doripenem, Imipenem/Cilastatin or Meropenem.

In further embodiments, the antibiotic agent may be a cephalosporin (first generation) such as Cefadroxil, Cefazolin, Cefalexin, Cefalotin or Cefalothin, or alternatively a Cephalosporin (second generation) such as Cefaclor, Cefamandole, Cefoxitin, Cefprozil or Cefuroxime. Alternatively the antibiotic agent may be a Cephalosporin (third generation) such as Cefixime, Cefdinir, Cefditoren, Cefoperazone, Cefotaxime, Cefpodoxime, Ceftibuten, Ceftizoxime and Ceftriaxone or a Cephalosporin (fourth generation) such as Cefepime and Ceftobiprole.

In other embodiments, the antibiotic agent may be a lincosamides such as Clindamycin and Azithromycin, or a macrolide such as Azithromycin, Clarithromycin, Dirithromycin, Erythromycin, Roxithromycin, Troleandomycin, Telithromycin and Spectinomycin.

In further embodiments, the antibiotic agent may be a monobactams such as Aztreonam, or a nitrofuran such as Furazolidone or Nitrofurantoin.

In other embodiments, the antibiotic agent may be a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In further embodiments, the antibiotic agent may be a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, and Trimethoprim-Sulfamethoxazole (Co-trimoxazole) (TMP-SMX).

In further embodiments, the antibiotic agent may be a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

The following examples are set forth to assist in understanding the invention and should not, of course, be construed as specifically limiting the invention described and claimed herein. Such variations of the invention, including the substitution of all equivalents now known or later developed, which would be within the purview of those skilled in the art, and changes in formulation or minor changes in experimental design, are to be considered to fall within the scope of the invention incorporated herein.

### Example 1—Inhaled MMP Inhibitor (Compound 1) is Absorbed by the Mouse Lung

Studies were conducted to determine the potential efficacy of butanamide, N-hydroxy-2-[[(4-hydroxyphenyl)sulfonyl](3-pyridinylmethyl)amino]-3-methyl, hydrochloride, (2R) (CAS: 779342-04-0, assay 95%, hereinafter referred to as “Compound 1”) a non-specific MMP inhibitor as an inhaled therapy in the mitigation of acute inflammatory lung injury. We first assessed if Compound 1 could be effectively delivered to the lung via inhalation. Compound 1 was obtained from Nanjing Kaimubo Pharmatech Company Limited (formerly Chembo Pharma Company Limited) at 789th Hushan Road, Jiangning District, Nanjing, China.

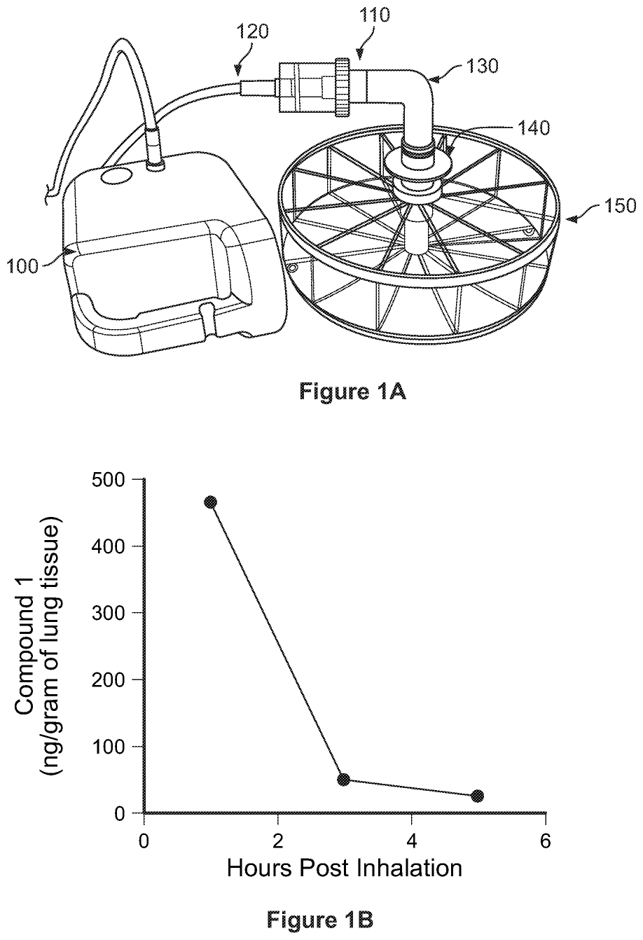

The compound was administered with a nebulizer of (FIG. 1A). With a 9 cc Medication Nebulizer, a dose of liquid medicine was prepared to a final concentration of 5 ml (3 mg/mL). Treatment was for approximately 20-30 minutes until all liquid was delivered. Nebulization did not require oxygen. The nebulizer includes a Hospital grade Schuco air compressor 100, Blue top medication nebulizer 110, Plastic collar 120, White elbow hose connector 130, 7′ crush proof 02 tubing to air compressor 140, and MPC Aerosol Pie Cage 150. Technical Specifications of the nebulizing system were as follows:

Compressor pressure . . . 35-45 psig
High compressor operating flow rate . . . 7-11 lpm
Therapeutic particle size . . . 0.5 um-5 um.

Based on literature, there was oral delivery of 100-500 mg/kg or 50-300 mg/60 kg human dose (a dose-finding and pharmacokinetic study of the matrix metalloproteinase inhibitor MM1270 (previously termed CGS27023A)) with 5-FU and folic acid). Therefore, 50 mg/kg/day would be recognized as a safe dose.

The pie cage 150 has 12 slots to hold 12 mice. Assuming each mouse has exposure to 1/12^{th} of the nebulized drug, the nebulizer needs 15 mg of Compound 1 to administer each mouse a dose of 50 mg/kg/day (50 mg/kg*12*0.025 kg=15 mg).

To test whether the nebulized drug (Compound 1) is absorbed in the lung and BAL samples. 10 mice were divided into three trial groups:

Low dose group: 3 mice at 2 mg dose (˜5 mg/kg)
Medium dose group: 3 mice at 5 mg dose (˜12 mg/kg)
High dose group: 4 mice at 10 mg dose (˜35 mg/kg)

The mice were nebulized within their respective dosing group for fifteen minutes (starting with the lowest dosing and moving up to the highest). One mouse was sacrificed per group at 1 hour, 3 hours and 5 hours after the drug delivery (2 mice were sacrificed for the 10 mg dose group).

Five samples were collected during sacrifice: upper and lower left lung, right lung, and serum. The order of sample collection was: first blood, then left lung, then right lung. Before removing the right lung, but after removing the left lung, the right lung was lavaged. The lung samples were immediately placed into liquid nitrogen and the blood samples rested for 4 hours at room temperature. After the blood clotted it was centrifuged and the serum was taken into a new tube. Lung and serum samples were then analyzed by mass spectroscopy. Table I shows the lung and serum concentration of Compound I in the treated mice. FIG. 1B shows a graphical representation of the medium dose group data.

TABLE I

| | | | Mouse | Mouse |
|---|---|---|---|---|
| | | | Lung | Serum |
| Subject | | | MMI 270 | MMI 270 |
| ID | Treatment | Matrix | (ng/g) | (ng/mL) |
| L11 LL | Low Dose | Left Lung Lower | 102.10 | |
| | 1 hour later | Lobe | | |
| L32 LL | Low Dose | Left Lung Lower | 42.92 | |
| | 3 hour later | Lobe | | |
| L53 LL | Low Dose | Left Lung Lower | 13.21 | |
| | 5 hour later | Lobe | | |
| M11 LL | Medium Dose | Left Lung Lower | 465.18 | |
| | 1 hour later | Lobe | | |
| M32 LL | Medium Dose | Left Lung Lower | 49.60 | |
| | 3 hour later | Lobe | | |
| M53 LL | Medium Dose | Left Lung Lower | 26.04 | |
| | 5 hour later | Lobe | | |
| H11 LL | High Dose | Left Lung Lower | 1415.95 | |
| | 1 hour later | Lobe | | |
| H32 LL | High Dose | Left Lung Lower | 119.51 | |
| | 3 hour later | Lobe | | |
| H53 LL | High Dose | Left Lung Lower | 70.67 | |
| | 5 hour later | Lobe | | |
| L11 RL | Low Dose | Right Lung Lower | 155.55 | |
| | 1 hour later | Lobe | | |
| L32 RL | Low Dose | Right Lung Lower | 12.60 | |
| | 3 hour later | Lobe | | |
| L53 RL | Low Dose | Right Lung Lower | <LLOQ | |
| | 5 hour later | Lobe | | |
| M11 RL | Medium Dose | Right Lung Lower | 127.68 | |
| | 1 hour later | Lobe | | |
| M32 RL | Medium Dose | Right Lung Lower | 18.92 | |
| | 3 hour later | Lobe | | |
| M53 RL | Medium Dose | Right Lung Lower | 13.55 | |
| | 5 hour later | Lobe | | |
| H11 RL | High Dose | Right Lung Lower | 598.24 | |
| | 1 hour later | Lobe | | |
| H32 RL | High Dose | Right Lung Lower | 50.75 | |
| | 3 hour later | Lobe | | |
| H53 RL | High Dose | Right Lung Lower | 23.36 | |
| | 5 hour later | Lobe | | |
| L11 S | Low Dose | Serum | | 2.97 |
| | 1 hour later | | | |
| L32 S | Low Dose | Serum | | <LLOQ |
| | 3 hour later | | | |
| L53 S | Low Dose | Serum | | <LLOQ |
| | 5 hour later | | | |
| M11 S | Medium Dose | Serum | | 20.06 |
| | 1 hour later | | | |
| M32 S | Medium Dose | Serum | | 3.40 |
| | 3 hour later | | | |
| M53 S | Medium Dose | Serum | | <LLOQ |
| | 5 hour later | | | |
| H11 S | High Dose | Serum | | 87.99 |
| | 1 hour later | | | |
| H32 S | High Dose | Serum | | 7.93 |
| | 3 hour later | | | |
| H53 S | High Dose | Serum | | 3.79 |
| | 5 hour later | | | |

None of the doses (including the highest dose) caused mortality or observable toxicity.

As shown in Table I and in FIG. 1B, mice treated with Compound i demonstrated effective absorption of Compound i in the lung. Dose size had no significant effect on the concentration of Compound i at any of the time points examined. At i hours post-inhalation, the levels of Compound i were significantly increased compared to the 3 hours or the 5 hour time points. No significant different was seen between the 3 hours and the 5 hours time points.

Based on this data, the highest dose of 10 mg in the nebulizer (about 35 mg/kg of body weight) was used for future experiments summarized in Examples 2-4 (unless indicated otherwise).

### Example 2—Treatment with Inhaled Compound 1 Attenuates LPS-Induced Pulmonary Inflammation

We next examined if inhaled Compound 1 inhibited MMP activity in lungs of mice injured with lipopolysaccharide (LPS). Mice lungs injured with LPS mimic acute inflammatory lung injury in humans. Several studies in mice have examined the effects of LPS on pulmonary inflammation and injury as far 7 weeks post-exposure. Throughout the study period, increased BALF inflammatory cells counts and pulmonary edema were observed. Additionally, in those studies, alterations in collagen deposition were observed by 5 weeks post exposure, suggesting a single dose of LPS results in permanent lung injury. Given these data, we have determined that the murine single exposure LPS model examined over a one-month period is a suitable model to examine the effectiveness of the treatment methods described herein as it recapitulates an acute period of inflammation followed by lung remodeling as observed in patients experiencing the pulmonary conditions targeted herein.

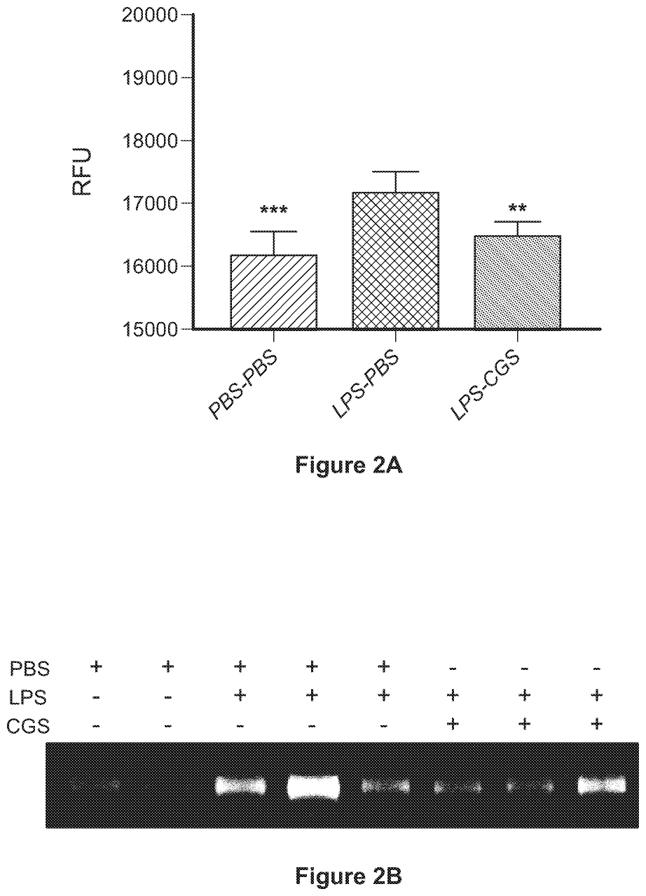

MMP 9 was selected as a target as it is a well-established mediator of inflammation in lung injury. 12-week-old male C57BL/6J mice were intranasally instilled with LPS (1.5 mg/kg of 1 mg/ml solution in sterile PBS). 24 hours later, these mice received a single dose of aerosolized MMP inhibitor (Compound 1, dissolved in PBS, 35 mg/kg) or PBS alone. One hour following nebulization, the mice were sacrificed and assessed for MMP activity in both the bronchoalveolar lavage fluid (BALF) and lung tissue homogenate obtained from treated mice. One hour following nebulization of Compound 1, the presence of the aerosolized Compound 1 in the lung was confirmed at concentrations >100 ng/g of tissue. MMP activity in BALF was determined using a commercially available MMP 9 Quantikine assay from R&D systems. As shown in FIG. 2A, Compound 1 inhibits MMP 9 activity in the BALF. Lung homogenate was assessed using gel zymography and display a similar decrease in MMP 9 activity (FIG. 2B).

### Example 3—Inhaled Compound 1 Attenuates Features of Acute Inflammatory Lung Injury Observed in Mice Lungs Injured with LPS (Such as Inflammation Score, Edema, Apoptosis)

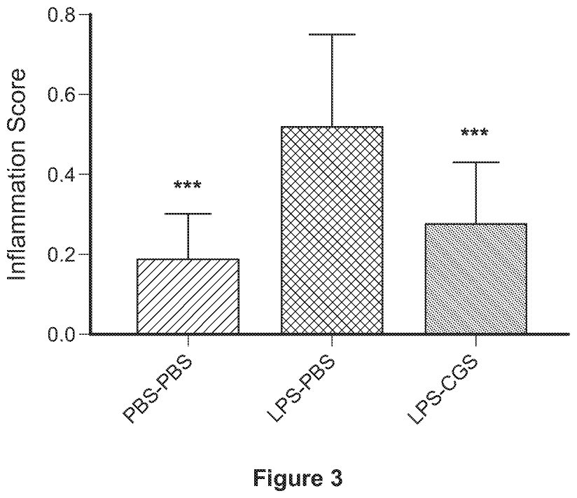

We next determined if inhaled Compound 1 attenuated specific features of acute inflammatory lung injury that were also observed in response to LPS. Again, 12-week-old male C57BL/6J mice were intranasally instilled with LPS (1.5 mg/kg of 1 mg/ml solution in sterile PBS). 24 hours later, these mice received a single dose of aerosolized MMP inhibitor (Compound 1, dissolved in PBS, 35 mg/kg) or PBS alone and a tail vein injection of FITC labeled albumin (FITC-albumin). One hour following nebulization, the mice were sacrificed. The left lung was fixed in formalin from each animal, paraffin embedded, sectioned and stained with H&E to examine histological features indicative of lung injury. These were achieved by visually scoring each slide based on a lung injury scoring system developed by the American Thoracic Society for animal models of inflammatory lung injury. As shown in FIG. 3, Compound 1 treatment reduces the lung injury score (also referred to as “Inflammation Score”) in response to LPS treatment with Compound 1 (***p<0.001, significantly different from LPS alone).

Tissue edema was assessed using two methods. First, serum and BALF fluid were collected from mice and FITC-albumin levels were measured in each using a fluorescent microplate reader. Fluid leakage into injured lungs is indicative of increased vascular permeability and could be indicative of edema. A greater fluid leakage would also be characterized by increased levels of FITC-albumin in the BALF. To determine the level of vascular permeability and edema, the ratio of BALF FITC-albumin to serum FITC-albumin was calculated. An increase in the ratio of BALF FITC-albumin to serum FITC-albumin is indicative of higher levels of permeability and edema.

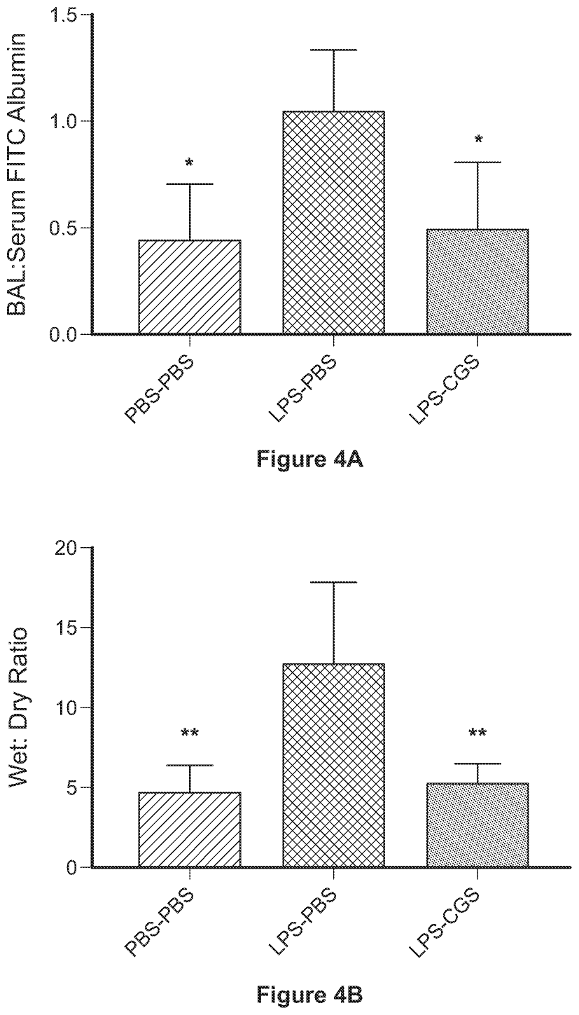

As shown in FIG. 4A, Compound 1 treatment restored pulmonary vascular permeability as compared to mice only receiving LPS (0.44 for control, 0.49 for Compound 1 vs 1.1 for LPS, n=6/group, * p<0.05). FIG. 4A demonstrates that Compound 1 blocks inflammation related edema in the lung.

Edema was also assessed by determination of pulmonary wet to dry ratios. At the time of sacrifice the right middle lobe was removed from each animal and promptly weighed to obtain the wet weight. Thereafter, the lung tissue was dried in an oven at 65° C. for 72 hours and then weighed to obtain the dry weight. The wet weight and the dry weight were used to calculate the wet:dry ratios summarized in FIG. 4B.

As shown, Compound 1 treatment reduces pulmonary edema to control levels as compared to LPS treated mice (4.1 for control, 7.3 for Compound 1 vs 12.9 for LPS, n=3/group, ** p<0.01).

The wet:dry ratio results depicted in FIG. 4B concur with the FTIC-albumin results depicted in FIG. 4A.

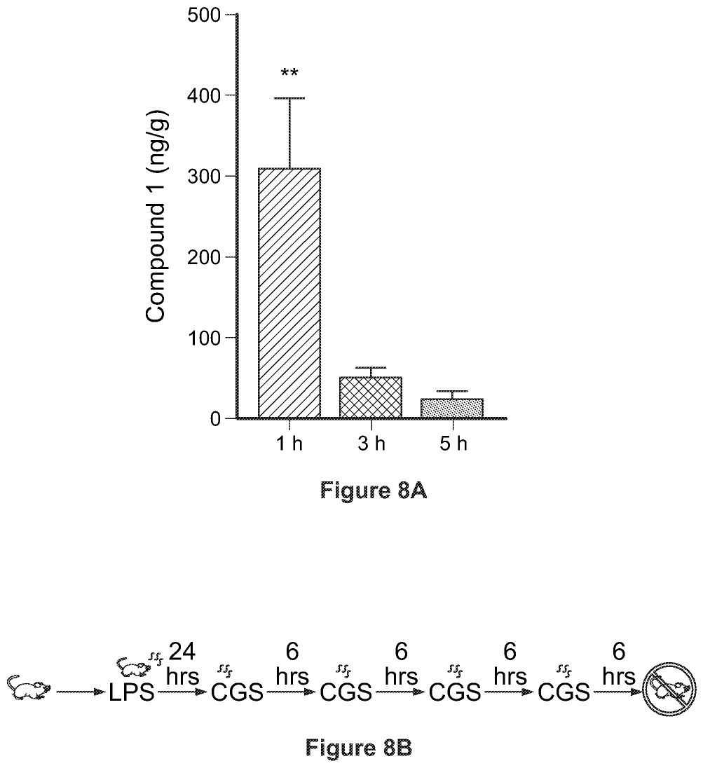

It was also examined if Compound 1 would reduce the levels of apoptosis associated with injury by LPS. Because the time line of apoptosis takes hours, an additional set of mice were injured with LPS (1.5 mg/kg of 1 mg/ml solution in sterile PBS) followed by initiation of treatment with Compound 1 twenty four hours later. The mice received four doses of Compound 1 (one dose every six hours over a total duration of 24 hours in accordance with the treatment schedule described below with reference to FIG. 8B). The mice were sacrificed and their lungs were harvested and assessed for caspase 3 activity by western blot for cleaved caspase 3 and a caspase 3/7 activity assay obtained from ThermoFisher.

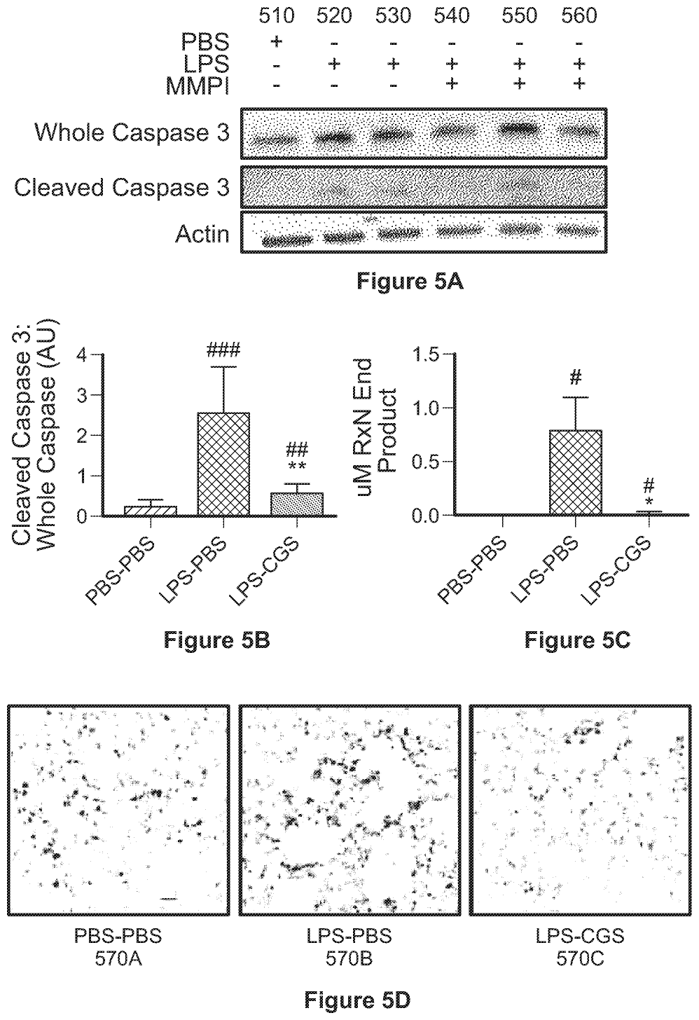

FIG. 5A is a representative western blot of lung homogenates that were equalized for protein content (i.e., normalized to actin) and probed for whole and cleaved caspase 3. Each lane represents a lung homogenate sample (40 μg) from an individual mouse. Lane 510 depicts a lung homogenate sample from a control mouse that was not administered LPS or Compound 1. Lanes 520 and 530 depict lung homogenate samples from two mice that were administered LPS but were not treated with Compound 1. Lanes 540, 550, and 560 depict lung homogenate samples from three mice that were administered LPS and treated with Compound 1. As can be seen from FIG. 5A, LPS treatment increased levels of cleaved caspase-3, indicating LPS induced apoptosis. In contrast, caspase 3 activation was absent or reduced in mice treated with LPS and Compound 1, with some expected variation among animals.

FIG. 5B is the relative densitometric quantification of western blots for cleaved caspase 3: whole caspase ratio. As shown, LPS treatment increased the cleaved:whole caspase 3 ratio as compared to control animals (2.57±1.15 AU for LPS-PBS and 0.59±0.18 AU for LPS-CGS vs. 0.25±0.15 AU for PBS-PBS; n≥5). This data demonstrates that Compound 1 inhaled treatment attenuated the LPS-induced increase in cleaved caspase 3. FIG. 5C depicts the results of the caspase 3/7 activity assay performed on mouse lung homogenate. For both graphs: ^{###}p<0.001, ^{##}p<0.01, ^{#}p<0.05, significantly different from control; *p<0.01, *p<0.05, significantly different from LPS-PBS; n≥8 for all groups. In agreement with western blot analysis (FIGS. 5A, 5B), LPS treatment increased caspase 3/7 activity significantly as compared to PBS controls (0.81±1.15 μM for LPS-PBS and 0.05±0.16 μM for LPS-CGS vs. 0.00±0.00 μM for PBS-PBS; n≥4). The increase in caspase 3/7 activity was attenuated by inhaled treatment with Compound 1. FIG. 5D depicts 20× images (570A, 570B, and 570C) of representative sections of lung tissue fluorescently stained for cleaved caspase-3 (red) and cell nuclei (blue) in a control lung tissue (570A), a lung tissue of a mouse treated with LPS only (570B), and lung tissue of a mouse treated with LPS and Compound 1 (570C) (n=3).

All data demonstrated a pronounced attenuation of caspase-3 activity and a corresponding decrease in apoptosis in lungs receiving inhaled Compound 1 following LPS injury.

### Example 4—Compound 1 Attenuates Neutrophil Infiltration

Since substantial literature indicates neutrophils (PMN) are critical mediators of inflammatory damage in injured lung, we next assessed if inhaled Compound 1 attenuated PMN infiltration of LPS injured lungs. To achieve this, mice were injured with LPS and 24 h later received a single inhaled dose of Compound 1 and were sacrificed 1 hour after dosing. PMN infiltration was assessed by examining lung sections (qualitatively) and BALF (quantitatively) from Compound 1 treated and untreated mice.

Quantitative examination was performed on BALF samples that were collected from each animal and subsequently subjected to cytospin centrifugation and DifQuik staining. The fraction of PMN present in each sample may be quantified by counting 300 cells of the inflammatory cell population across three different microscopic images. The obtained fraction may then be multiplied by the total cell count to estimate the number of PMNs present in the BALF.

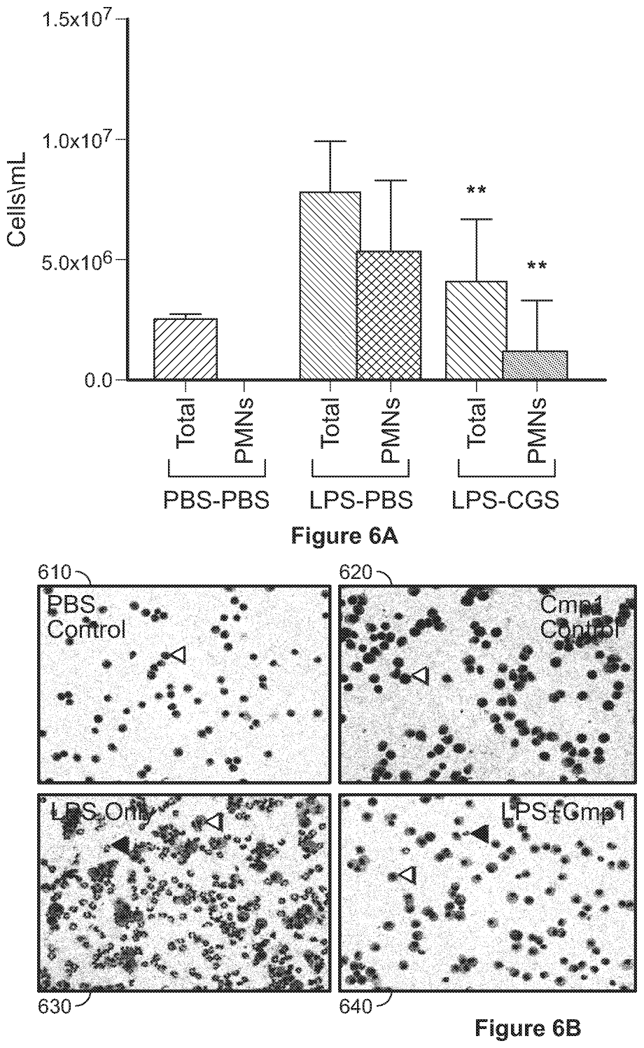

As shown in FIG. 6A, 1 hour following treatment Compound 1 significantly reduced total PMN counts in the BALF for LPS treated mice (5.3×10⁶ neutrophils/ml in LPS/PBS treated vs. 1.2×10⁶ neutrophils/ml in LPS/Compound 1 treated, n=5/group p=0.01). The macrophage count in the BALF for LPS treated mice remained unchanged. Treatment with Compound 1 also resulted in a significant reduction in the overall cell count in LPS treated mice. Lung sections revealed decreased inflammation and attenuation of the LPS lung injury in Compound 1 treated mice.

FIG. 6B displays representative images of stained cytospins from each experimental group (610-PBS control group, 620-Compound 1 control group, 630-LPS treated group, 640-LPS+Compound 1 treated group). LPS treated animals that received inhaled Compound 1 are characterized by significantly decreased presence of PMNs in the BALF (blue arrows, macrophages; red arrows, PMNs).

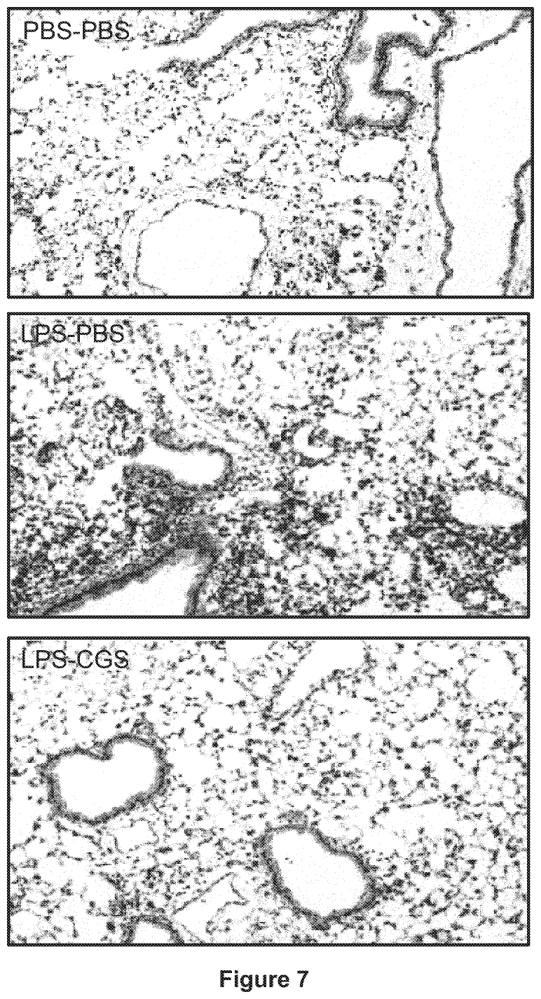

To assess the effects of Compound 1 on PMN in situ, paraffin embedded lung sections (prepared as described previously) were stained for myeloperoxidase, a PMN marker. As shown in FIG. 7, treatment with Compound 1 attenuates PMN infiltration into the lung parenchyma in LPS treated animals.

### Example 5—Alternative Treatment Schedule

FIG. 8A demonstrates, similar to FIG. 1B, that inhaled Compound 1 is successfully absorbed by the mouse lung and that inhalational delivery achieves effective absorption of Compound 1 in the lungs at low concentrations. MMP inhibitors, initially developed as chemotherapeutic agents, failed previous clinical trials due to lack of clinical efficacy or intolerable side effects. These were observed with chronic, systemic administration required for cancer treatment (Alaseem A, et al. Matrix Metalloproteinases: A challenging paradigm of cancer management. Semin Cancer Biol. 2019; 56). We determined whether circumventing systemic administration could be achieved via inhalational delivery of the non-specific MMP-inhibitor, such as Compound 1. This method permits delivery at lower doses than required parenterally, reducing the likelihood of side effects.

In this test, C57/BL6 mice were divided into three treatment groups, where they received nebulized Compound 1 at high dose (10 mg/kg), medium dose (5 mg/kg) and, low dose (2 mg/kg). Animals were sacrificed at 1, 3 and, 5 hours (n=3 mice per time point) followed by collection of lung homogenates. Homogenates were analyzed by mass spectroscopy to determine Compound 1 levels in the lung. Dose size had no significant effect on Compound 1 concentration at any of the time points examined. At 1 hour post-inhalation Compound 1 levels were significantly higher as compared to the 3 hours or 5 hours time points while no difference was found between the 3 hours and 5 hours groups (310.7±85.38 ng/g at 1 h vs 49.03±15.54 ng/g and 24.48±9.96 ng/g at 3 and 5 h, respectively; n=6; *p<0.01).

The data suggests that the concentration of Compound 1 in the mouse lung was dependent on time rather than dose. Therefore, the medium dose (5 mg/kg) was administered in experiments described hereinafter (unless indicated otherwise).

Based on the data, the effect of a 6 hour dosing interval (q6h) of Compound 1 on injured mice lung was explored. FIG. 8B schematizes the experimental LPS injury/Compound 1 treatment workflow utilized unless otherwise indicated. Briefly, mice were nasally instilled with LPS (1.5 mg/kg) followed by initiation of Compound 1 treatment 24 hours later (i.e., after a 24 hours rest period). Four doses of Compound 1 (12 mg/kg) were administered via nebulizer every 6 hours to maintain a constant presence of compound in the lung. PBS was administered as a control. Mice were sacrificed 6 hours following the fourth dose.

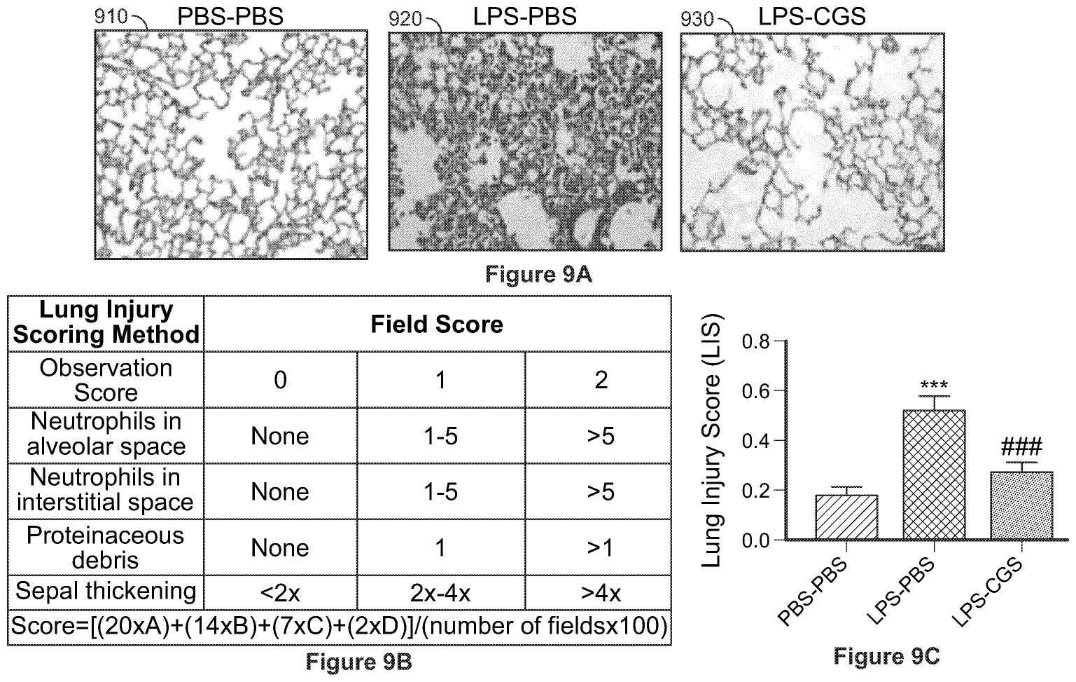

It was next determined if the proposed treatment schedule was efficacious in ameliorating LPS induced pulmonary inflammation. Substantial evidence indicates MMPs mediate portions of the pulmonary inflammatory cascade ranging from secretion and activation of cytokines to tight junction permeability in addition to their canonical roles in matrix degradation and cell migration (Fligiel S E, et al. Matrix metalloproteinases and matrix metalloproteinase inhibitors in acute lung injury. Hum Pathol. 2006; 37(4); Becker-Pauly C, et al. TNFalpha cleavage beyond TACE/ADAM17: matrix metalloproteinase 13 is a potential therapeutic target in sepsis and colitis. EMBO Mol Med. 2013; 5(7); and Sapoznikov A, et al. Early disruption of the alveolar-capillary barrier in a ricin-induced ARDS mouse model: neutrophil-dependent and -independent impairment of junction proteins. Am J Physiol Lung Cell Mol Physiol. 2019; 316(1)). Qualitative visual inspection of H&E stained paraffin embedded sections (5 μM) revealed reduced inflammation in Compound 1 treated animals (930 in FIG. 9A) as compared to mice only receiving LPS (920 in FIG. 9A). Increased inflammatory infiltrate is apparent in animals receiving LPS followed by PBS (920 in FIG. 9A) as compared to animals receiving PBS only (910 in FIG. 9A).

To quantify these findings, we adapted a lung injury scoring (LIS) method previously published by Matute-Bell et al (2008). Slides were blinded and ≥5 20× field scores were captured per sample. Values were entered into the lung injury score equation (FIG. 9B) to obtain the final LIS for each mouse (re:lung injury score equation, see Amendola R S, et al. ADAM9 disintegrin domain activates human neutrophils through an autocrine circuit involving integrins and CXCR2. J Leukoc Biol. 2015; 97(5)).

LPS only controls (0.524±0.054 LIS, n=5, ***p<0.001) were characterized by substantially increased inflammation (LIS) as compared to PBS only controls (0.188±0.025 LIS, n=5; FIG. 9C). In contrast, treatment with inhaled Compound 1 following LPS injury resulted in a significantly decreased LIS (0.277±0.031 LIS vs 0.524±0.054 LIS, respectively; n=5; ^{###}p<0.001). A total of 5 fields per section were analyzed for each mouse.

Interestingly, field scores showed marked reduction in both neutrophil subcategories (i.e., neutrophils in the alveolar space and neutrophils in interstitial space). This demonstrates an association between neutrophil counts and ALI/ARDS severity.

### Example 6—Compound 1 Inhaled Treatment Based on the Alternative Treatment Schedule Attenuated the Development of LPS-Induced Pulmonary Edema and Increase in Vascular Permeability

A major feature of ALI/ARDS is non-cardiogenic pulmonary edema resulting in profound hypoxemia. The previously presented data indicates that inhaled Compound 1 mitigates the inflammatory effects of LPS via anatomic assessment. We next investigated if attenuation of edema occurred with the alternative treatment schedule where LPS-injured mice were treated with Compound 1 every 6 hours for 4 doses.

Total lung edema was determined by assessing lung wet:dry ratios under each experimental condition. Following completion of Compound 1 treatment, the right lung was removed and immediately weighed followed by desiccation for 72 hours and weighed a second time to determine the dry weight. Wet:dry weight ratios were then calculated.

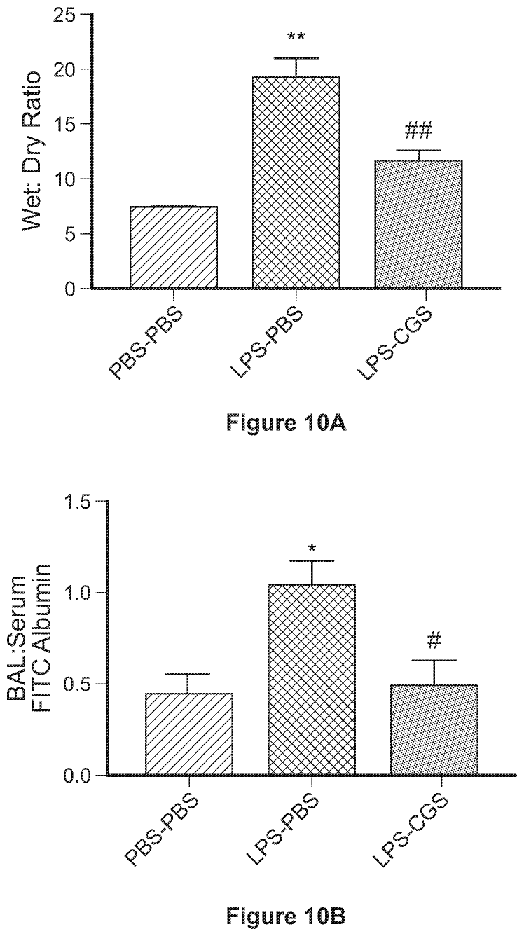

As shown in FIG. 10A, injury with LPS alone resulted in a significant increase in the wet:dry weight ratio (19.44±1.6; n=5, *p<0.01) as compared to PBS only controls (7.5±0.12; n=5), confirming LPS injury results in pulmonary edema. This was significantly reduced by Compound 1 treatment following LPS injury (11.80±0.86; n=5; ^{##}p<0.01). This demonstrated that Compound 1 treatment significantly attenuated the LPS-induced increase in edema (19.44±1.6 in LPS alone group vs. 11.80 i 0.86 in LPS followed by Compound 1 treatment group; n=5, *p<0.01, ^{##}p<0.01).

It was next determined if LPS induced edema was a result of increased permeability of the pulmonary vascular barrier. This was achieved using a FITC-albumin exclusion assay. Approximately 1 hour prior to sacrifice, mice received a tail vein injection of 3 mg of FITC labeled albumin. BALF and serum were collected and the amount of labeled albumin in each sample (BALF FITC Albumin:serum FITC Albumin) was determined using a fluorescent plate reader.

As shown in FIG. 10B, treatment with LPS significantly increased the BALF:serum ratio of FITC-albumin as compared to PBS controls (1.04±0.13 vs 0.44±0.11, respectively; n=5; *p<0.05), indicating the observed edema results from an increase in vascular permeability. Importantly, treatment of LPS injured mice with inhaled Compound 1 significantly decreased the FITC-albumin BALF: serum ratio as compared to mice receiving LPS alone (0.49±0.13 vs 1.04±0.13, respectively; n=5; *p<0.05; ^{#}p<0.05).

Taken together, these data demonstrate that LPS induced pulmonary edema results from vascular barrier breakdown and, the ability of Compound 1 to restore barrier integrity and reduce edema in the injured lung supports MMPs as mediators of this process.

### Example 7—Compound 1 Inhaled Treatment Based on the Alternative Treatment Schedule Attenuated the Influx of Neutrophils into LPS-Injured Lungs

One of the earliest events in inflammatory 1 mg injury is acute neutrophil influx. Neutrophil migration is driven by myriad cytokines and chemokines produced in response to tissue injury (Zemans, 2009 #69) (Adams J M, et al. Early trauma polymorphonuclear neutrophil responses to chemokines are associated with development of sepsis, pneumonia, and organ failure. J Trauma. 2001; 51(3); Abraham E. Neutrophils and acute lung injury. Crit Care Med. 2003; 31(4 Suppl)) several of which are known targets of MMPs (Lee K S, et al. Matrix metalloproteinase inhibitor regulates inflammatory cell migration by reducing ICAM-1 and VCAM-1 expression in a murine model of toluene diisocyanate-induced asthma. J Allergy Clin Immunol. 2003; 111(6); Vandenbroucke R E, et al. Matrix metalloproteinase 13 modulates intestinal epithelial barrier integrity in inflammatory diseases by activating TNF. EMBO Mol Med. 2013; 5(7)).

Given that Compound 1 treatment lowered the LIS by preferentially effecting neutrophil levels (see FIGS. 9A,B,C), we more closely examined the effects of MMP inhibition on neutrophil influx.

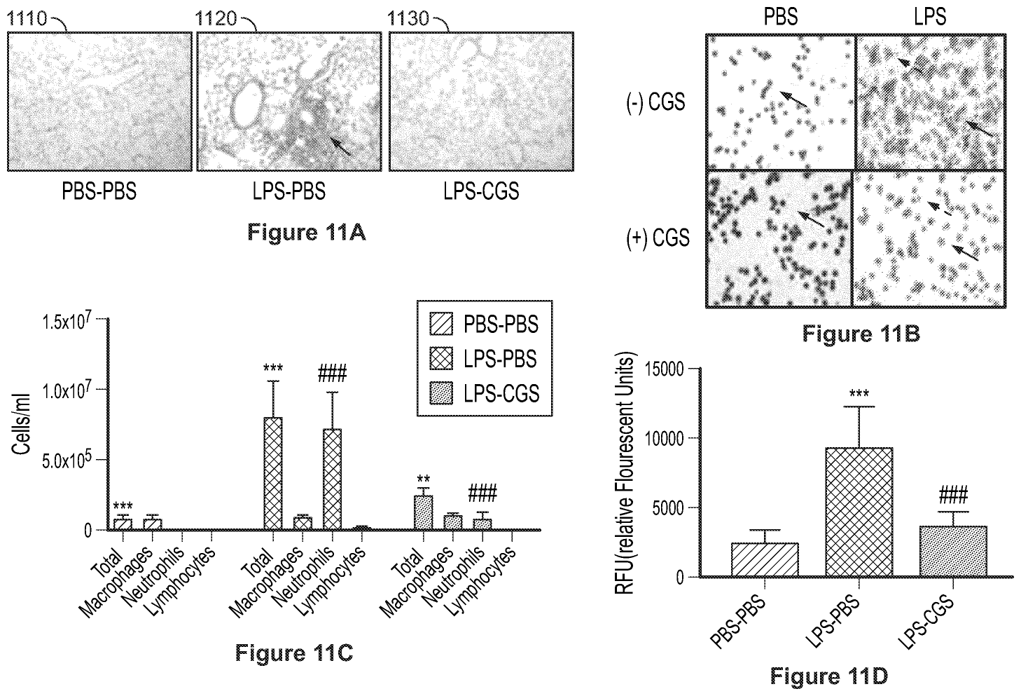

Mice were nasally instilled with LPS followed by treatment with CGS every 6 hours for 4 doses. Mice were sacrificed 6 hours after the 4^{th} dose of Compound 1 and lungs and BALF were collected for analysis. Paraffin embedded lung sections (5 μM) were stained for myeloperoxidase, a neutrophil marker, followed by detection using DAB, to assess parenchymal neutrophil infiltration. As shown by representative sections in FIG. 11A, significantly more myeloperoxidase positive cells (brown; black arrow) were present in LPS-PBS animals (1120) as compared to PBS controls (1110) or those receiving Compound 1 (1130) (10× images). This demonstrates that Compound 1 treatment qualitatively reduced parenchymal neutrophil infiltration in response to LPS.

Quantitative examination of neutrophil infiltration was performed on DifQuik stained cytopsins of BALE Representative DifQuik stains in FIG. 11B clearly demonstrate Compound 1 inhaled treatment decreases BALF neutrophil counts (red arrows) following treatment of LPS-injured mice as compared to LPS animals (green arrows are macrophages).

Differential cell counts were taken from each animal's cytospin with at least 3 separate images counted. As shown in FIG. 11C, this observation is manifest as a significant decrease in the overall inflammatory cell count in LPS-injured animals receiving inhaled Compound 1 treatment as compared to their LPS only treated counterparts (8.6e5±3.7e5 cells/mL vs 8.0e6±5.7e6 cells/mL, respectively; n≥4; **p<0.01, ***p<0.001, ^{###}p<0.001).

In agreement with DifQuik staining, quantification of neutrophils in the BALF revealed a substantial decrease in the neutrophil count within the BALF of Compound 1 treated animals as compared to LPS alone (8.0e6±5.7e6 cells/ml for LPS-PBS vs 2.4e6±1.2e6 cells/ml for LPS-CGS; n=≥5).

Inhibition of MMP-9 by inhaled Compound 1 treatment was assessed using a Quantikine Assay (R&D systems). Mice were injured by nasal instillation of LPS followed by treatment with inhaled PBS or Compound 1 (5 mg/mL). One-hour post-Compound 1 treatment lung homogenates were collected and analyzed. LPS significantly increased MMP-9 activity as compared to PBS only controls as determined by an MMP-9 Quantikine ELISA kit (R&D Systems; 9260±1173 RFU vs 2388±370 RFU, respectively; n=6; ***p<0.001; ^{###}p<0.001). Treatment with Compound 1 significantly reduced the LPS-mediated increase in MMP-9 activity (3614±442.1 RFU vs 9260 RFU±1173 RFU, respectively; n=6 per group; ^{###}p<0.001).

Furthermore, MMP-9 levels decreased significantly in whole lung homogenate from Compound 1 treated animals (FIG. 11C). This finding lends support to the observed decrease in neutrophil infiltration since increased MMP-9 expression is a well demonstrated component of the neutrophil inflammatory response (Bradley L M, et al. Matrix metalloprotease 9 mediates neutrophil migration into the airways in response to influenza virus-induced toll-like receptor signaling. PLoS Pathog 2012; 8(4); Keck T, et al. Matrix metalloproteinase-9 promotes neutrophil migration and alveolar capillary leakage in pancreatitis-associated lung injury in the rat. Gastroenterology. 2002; 122(1)).

### Example 8—Compound 1 Inhaled Treatment Based on the Alternative Treatment Schedule Inhibits Neutrophil Efflux from the Pulmonary Circulation into the Lung In Vivo Following LPS Injury

To examine the hypothesis that reduced neutrophil counts in response to Compound 1 treatment resulted from decreased cell migration from the pulmonary circulation, pulse-chase labeling of newly synthesized neutrophils in LPS exposed animals was performed. LPS or saline was nasally instilled into mice which were then allowed to recover for 24 hours. Following this, an intraperitoneal injection (IP injections) of bromodeoxyuridine (BrdU) was administered and animals were allowed to rest for 2.5 hours. A 2.5-hour rest period was selected since it was previously determined that BrdU labeled neutrophils take about 3 hours to reach and infiltrate the lungs. At 2.5 hours animals received inhaled Compound 1 or PBS and were sacrificed at either 15- or 45 minutes post-treatment. Cytospin slides were generated from BALF and immunofluorescently stained for BrdU and MPO to identify neutrophils. Confocal images were taken of cytospins stained for BrdU and MPO (myeloperoxidase). The number of BrdU (+) and BrdU (−) neutrophils was quantified.

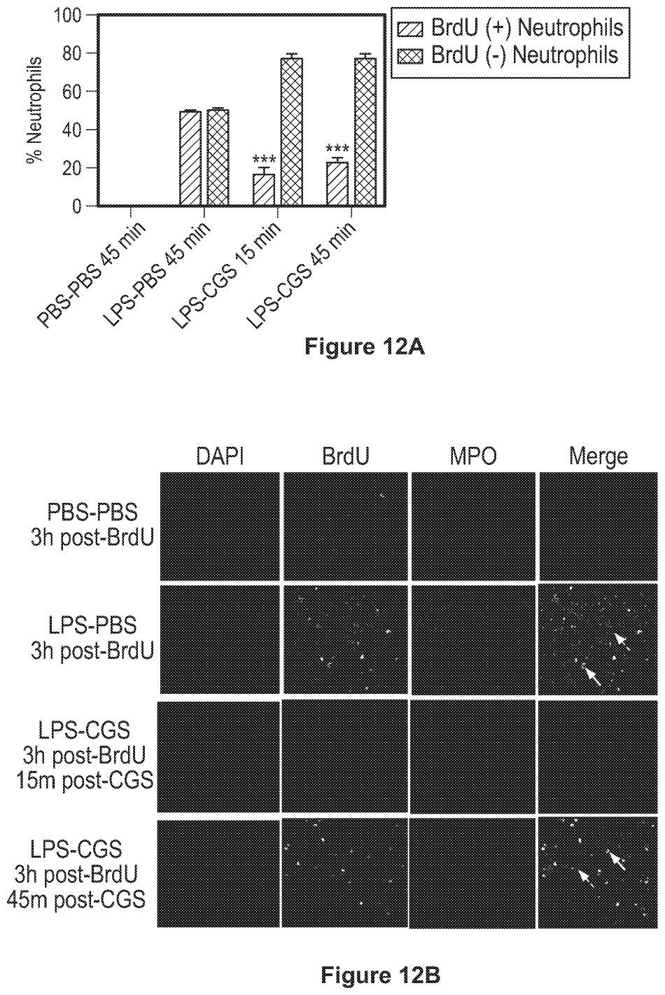

As shown in FIG. 12A, mice receiving LPS displayed equal proportions of BrdU positive and negative neutrophils. This indicated newly produced neutrophils were entering the lung. In contrast, as early as 15 minutes post-treatment mice receiving LPS followed by Compound 1 were characterized by a disproportionately low number of BrdU positive neutrophils. FIG. 12A demonstrates that Compound 1 inhaled treatment at 15 minutes and 45 minutes significantly decreased the percentage of BrdU (+) neutrophils in LPS treated mice as compared to LPS alone (16.7±3.6% at 15 minutes and 22.7±2.5% at 45 minutes versus 49.3±0.87% without Compound 1 treatment; 300 cells counted/cytospin, n=3; ***p<0.001).

FIG. 12B shows representative immunofluorescent images for stained cytospins of BALF from each condition showing BrdU stained neutrophils (red arrow) and macrophages (yellow arrow) with BrdU localized in the cytoplasma likely from phagocytized neutrophils.

PBS only animals lacked signal for myeloperoxidase (MPO) and BrdU. Interestingly, BrdU signal was also observed in the cytoplasm of macrophages (yellow arrows) suggesting they consume pulmonary neutrophils. Taken together, these data indicate Compound 1 treatment prevents the entry of neutrophils into the lung in response to LPS induced inflammation.

### Example 9—Compound 1's Migratory Inhibition in Murine Lungs is Relevant to Humans

Compound 1 significantly decreases neutrophil counts in LPS injured murine lungs potentially via migratory inhibition. To determine if this effect was relevant in humans we utilized a modified Boyden chamber to assess the effect of Compound 1 on migration of human neutrophils.

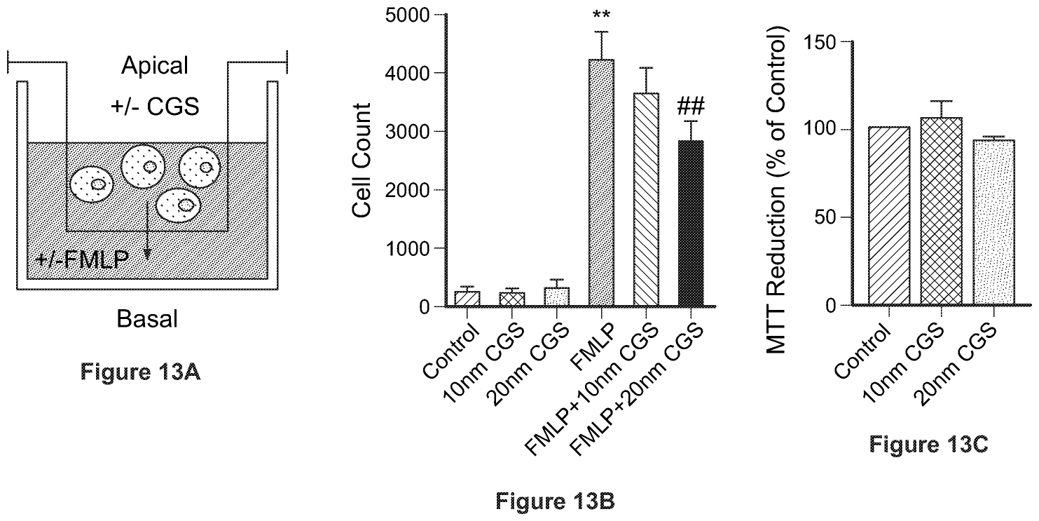

As shown in FIG. 13A, neutrophils were seeded into the apical chamber with or without the addition of Compound 1. The basal chamber was filled with media containing the chemoattractant N-Formylmethionyl-leucyl-phenylalanine (FMLP) or vehicle. The apical chamber was seeded with ˜100,000 neutrophils with the addition of Compound 1 or vehicle. In the basal chamber, the neutrophils were allowed to migrate for 1 hour after which the basal chamber was fixed, stained with DAPI, and photographed. Five fields per well were imaged and the number of neutrophils counted using NIH ImageJ.

As seen in FIG. 13B, FMLP treatment resulted in a significant increase in neutrophil migration into the basal chamber as compared to Compound 1 treated neutrophils and vehicle controls (4280±480 cells for FMLP vs 233±61 cells for 10 nm CGS vs 293±148 cells for 20 nm CGS; n=4; *p<0.01). Treatment with 20 nm Compound 1 significantly reduced neutrophil migration in response to FMLP (4280±480 cells for FMLP vs 2819±362 cells for FMLP+20 nm CGS; n=4; ^{##}p<0.01). FIG. 13B demonstrates that in all wells not treated with FMLP, minimal migration of neutrophils was observed into the basal chamber. FMLP treatment resulted in increased migration in a manner that was inhibited by Compound 1. This effect appeared to be dose-dependent but did not achieve significance at the lower 10 nm concentration of Compound 1.

To ensure the observed effects were not a result of reduced neutrophil viability, an MTT assay was utilized. Neutrophils were exposed to Compound 1 for 1 hour followed by addition of MTT to the culture medium for 30 minutes. Cells were then solubilized in DMSO and the absorbances were read on a microplate reader. At both the high and low concentrations, Compound 1 did not significantly alter neutrophil reductive capacity and has no significant effect on neutrophil viability in vitro (FIG. 13C).

Regulation of cell motility by the integrin β1 subunit is well-documented in multiple cell types, with several lines of evidence indicating MMPs play a significant role in expression and cellular localization of integrin β1 during neutrophil migration (Dumin J A, et al. Pro-collagenase-1 (matrix metalloproteinase-1) binds the alpha(2)beta(1) integrin upon release from keratinocytes migrating on type I collagen. J Biol Chem 2001; 276(31); Ugarte-Berzal E, et al. A 17-residue sequence from the matrix metalloproteinase-9 (MMP-9) hemopexin domain binds alpha4beta1 integrin and inhibits MMP-9-induced functions in chronic lymphocytic leukemia B cells. J Biol Chem 2012; 287(33)). To determine if MMP inhibition by Compound 1 interfered with integrin β1 expression in human neutrophils, cells were seeded onto glass chamber slides followed by Compound 1 treatment for 1 hour.

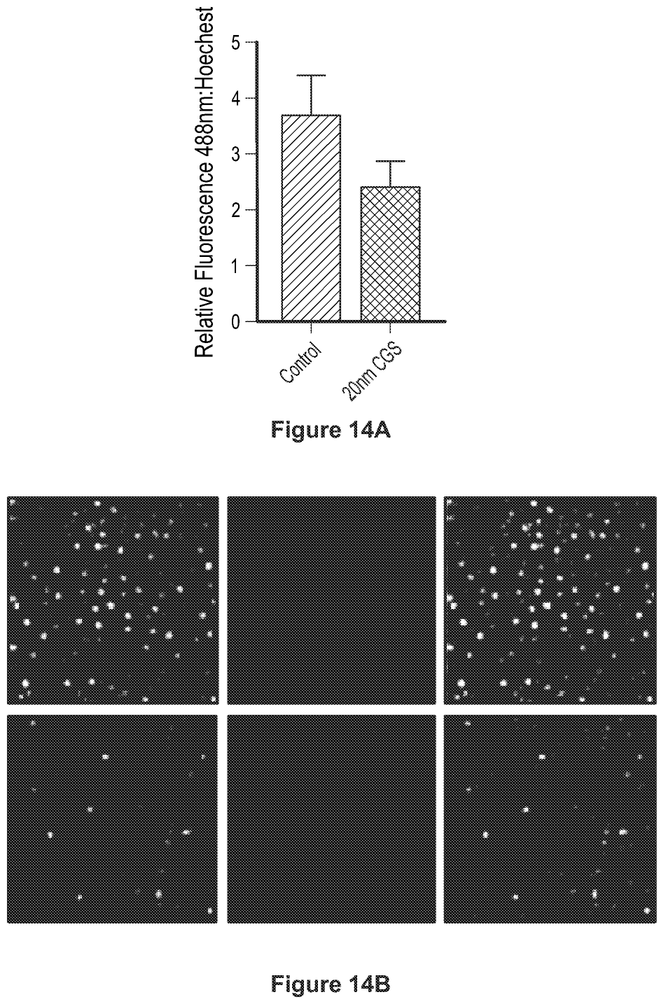

Cells were fixed and immunofluorescent staining was performed for integrin β1 and staining intensity was measured using NIH ImageJ and standardized to the number of cells per field. As shown in FIG. 14A, Compound 1 treatment decreased integrin 1 fluorescence intensity as compared to untreated controls (2.88±1.23 RFU vs 1.92±1.23 RFU, respectively; n=3; *p<0.05), consistent with the above data showing altered cell motility in response to FMLP. This demonstrates that Compound 1 treatment induced a significant decrease in integrin β1 expression.

### Example 10

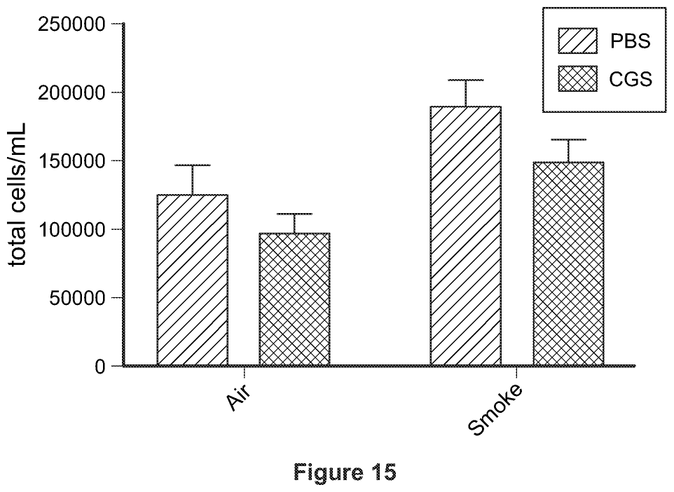

In order to determine if the observed effects of Compound 1 were specific to a model of acute lung injury, we exposed mice to cigarette smoke, a more chronic model of lung injury, for 10 days while treating once a day with inhaled Compound 1 for the last four days of smoke exposure. Mice were sacrificed 1 hour following their final dose of Compound 1 on the 10^{th} day. BALF was collected and total cell counts were performed. As shown in FIG. 15, Compound 1 failed to attenuate the smoke induced increase in BALF cell counts in this model system. (*p<0.05).

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present invention. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment

The present invention has been described with reference to specific exemplary embodiments thereof. The specification and drawings are, accordingly, to be regarded in an illustrative rather than a restrictive sense. Various modifications of the invention in addition to those shown and described herein will become apparent to those skilled in the art and are intended to fall within the scope of the appended claims.

1. A method of treating a pulmonary disease comprising administering via pulmonary delivery a therapeutically effective amount of a Matrix Metalloproteinase (MMP) Inhibitor to a patient in need thereof wherein the pulmonary disease is selected from the group consisting of Acute Respiratory Distress Syndrome, Acute Lung Injury and Acute Inflammatory Injury.

2. The method of claim 1, wherein the MMP inhibitor is a collagenase (MMP-1, MMP-8, MMP-13) inhibitor, gelatinases (MMP-2, MMP-9) inhibitor, stromelysins (MMP-3, MMP-10, MMP-11) inhibitor, matrilysins (MMP-7, MMP-26) inhibitor, membrane-type (MT) MMPs (MMP-14, MMP-15, MMP-16, MMP-17, MMP-24, MMP-25) inhibitor, and other MMPs (MMP-12, MMP-19, MMP-20, MMP-21, MMP-23, MMP-27, MMP-28) inhibitor, or a combination thereof.

3. The method of claim 2, wherein the MMP inhibitor is a collagenase MMP inhibitor.

4. The method of claim 1, wherein the MMP Inhibitor is a compound of Formula I: 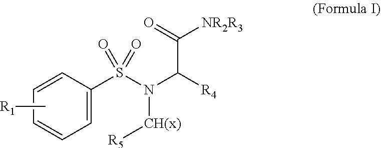
wherein X is an integer from 0-3;
R₁ is 0-3 substitutions independently selected from halogen, hydroxyl or C₁₋₃ alkyl;
R₂ and R₃ are independently H, hydroxyl or straight or branched C₁₋₃ alkyl;
R₄ is straight o branched C₁₋₅ alkyl; and
R₅ is a mono or bicyclic aromatic or heteroaromatic;
or pharmaceutically acceptable salt thereof or solvate thereof.

5. The method of claim 4, wherein the compound of Formula I is: 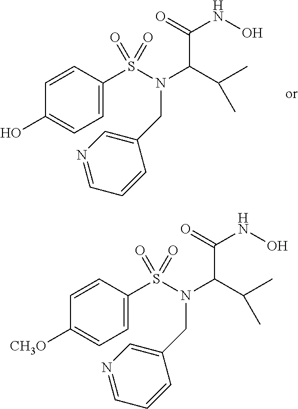
or pharmaceutically acceptable salt thereof or solvate thereof.

6. The method of claim 1, wherein the MMP Inhibitor is marimastat (BB-2516), batimastat (BB-94), PD166793, Ro32-3555, WAY170523, UK370106, TIMP1, TIMP2, TIMP3, TIMP4, RS113456, PKF242-484, CP 471,474, AZ11557272, AS112108, AS111793#, MMP408, GM6001 Ilomastat (Galardin®), doxycycline, R-94138, MMPI-I, MMPI-II (MMP2/MMP9 inhibitor II), MMP9 inhibitor I, MMP8 inhibitor I, ONO-4817, COL-3 (matastat), cyclohexylamine salt of (R)-1-(3′-methylbiphenyl-4-sulfonylamino)-methylpropyl phosphonic acid, MMI270, BMS-275291 (rebimastat), BAY 12-9566, SB-3CT, CH1104, or a combination thereof.

7. The method of claim 1, wherein the administration is pulmonary administration.

8. The method of claim 7, wherein the pulmonary administration is by oral inhalative administration or intranasal administration.

9. The method of claim 8, wherein the oral inhalative administration is by intratracheal instillation or intratracheal inhalation with an endotracheal tube.

10. The method of claim 8, wherein the intratracheal instillation comprises administering a solution or suspension of the MMP inhibitor to the pulmonary system by a syringe.

11. The method of claim 8, wherein the intratracheal inhalation comprises inhaling an aerosol comprising the MMP inhibitor.

12. The method of claim 11, wherein the aerosol is delivered by a metered dose inhaler.

13. The method of claim 8, wherein the intratracheal inhalation comprises inhaling a nebulized solution of the MMP inhibitor.

14. The method of claim 13, wherein the nebulized solution is delivered by jet nebulizer, ultrasonic nebulizer or vibrating mesh nebulizer.

15. The method of claim 8, wherein the intratracheal inhalation comprises inhaling a powder comprising the MMP inhibitor.

16. The method of claim 15, wherein the powder is administered by a dry powder inhaler.

17. A pharmaceutical composition comprising an effective amount of a Matrix Metalloproteinase (MMP) Inhibitor to for treating Acute Respiratory Distress Syndrome and a pharmaceutically acceptable excipient suitable for pulmonary administration.

18.-22. (canceled)

23. The pharmaceutical composition of claim 17, wherein the MMP Inhibitor is a compound of Formula I: 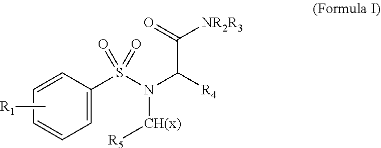
wherein X is an integer from 0-3;
R₁ is 0-3 substitutions independently selected from halogen, hydroxyl or C₁₋₃ alkyl;
R₂ and R₃ are independently H, hydroxyl or straight or branched C₁₋₃ alkyl;
R₄ is straight o branched C₁₋₅ alkyl; and
R₅ is a mono or bicyclic aromatic or heteroaromatic;
or pharmaceutically acceptable salt thereof or solvate thereof.

24. The pharmaceutical composition of claim 23, wherein the compound of Formula I is 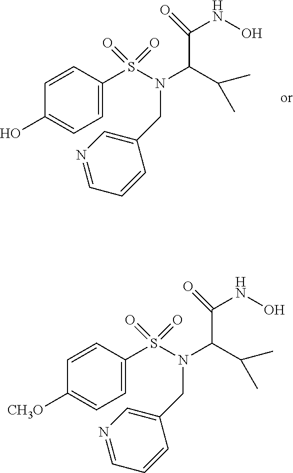
or pharmaceutically acceptable salt thereof or solvate thereof.

25. (canceled)

26. A drug delivery system comprising a pharmaceutical composition of claim 17 contained in a drug delivery device suitable for pulmonary administration.

27.-37. (canceled)
